(19) United States
(12) Patent Application Publication
(10) Pub. No.: US 2014/0274732 A1
(43) Pub. Date: Sep. 18, 2014

(54) METHODS AND COMPOSITIONS FOR NUCLEIC ACID SEQUENCING USING ELECTRONIC SENSING ELEMENTS

(71) Applicant: PACIFIC BIOSCIENCES OF CALIFORNIA, INC., Menlo Park, CA (US)

(72) Inventors: Jeremiah HANES, Menlo Park, CA (US); Jonas KORLACH, Newark, CA (US); Stephen TURNER, Menlo Park, CA (US)

(73) Assignee: PACIFIC BIOSCIENCES OF CALIFORNIA, INC., Menlo Park, CA (US)

(21) Appl. No.: 14/204,027

(22) Filed: Mar. 11, 2014

Related U.S. Application Data
(60) Provisional application No. 61/792,362, filed on Mar. 15, 2013.

(51) Int. Cl. C12Q 1/68

(52) U.S. Cl. CPC C12Q 1/6874
USPC 506/2

(57) ABSTRACT

The present invention is directed to methods, devices, compositions and systems for obtaining sequence data from nucleic acid templates by utilizing electronic sensing elements.

### CROSS-REFERENCE TO RELATED APPLICATIONS

This application claims priority to Provisional Application No. 61/792,362, filed on Mar. 15, 2013, the full disclosure of which is hereby incorporated in its entirety for all purposes.

### BACKGROUND OF THE INVENTION

Electronic devices and components have found numerous applications in chemistry and biology (more generally, “life sciences”), especially for detection and measurement of various aspects of chemical reactions and substance composition. Such electronic devices include ion-sensitive field effect transistors, often denoted in the relevant literature as ISFET (or pHFET). ISFETs conventionally have been explored to facilitate measurement of the ion concentration of a solution (for example hydrogen ion concentration or “pH”). Electronic devices can be of use in monitoring and detecting the products of numerous biological reactions, including nucleic acid hybridizations, protein-protein interactions, antigen-antibody binding, and enzyme substrate reactions, and have the advantage of favorable characteristics such as sensitivity, speed and miniaturization.

Many electronic detection systems in the detection of biological reactions are limited by the need for relatively high amounts of reagents and a low strength of signal, which can limit the amount and resolution of the information obtained from the reactions. There is thus a need for methods and compositions for increasing the signal generated in individual biological reactions to allow for the use of lower amounts of reagents and to increase the resolution of detection to the point of being able to monitor not only ensemble reactions of a synchronized population of molecules, but to also identify the products of individual single molecule reactions.

### SUMMARY OF THE INVENTION

Accordingly, the present invention provides methods and compositions for obtaining sequence data from nucleic acid templates. In some aspects, the methods generally comprise stepwise electronic sequence of a plurality of template nucleic acids. In other aspects, the methods comprise real-time single-molecule sequencing. In general, the methods involve detecting a signal that is associated with the cleavage of polyphosphate chains released from nucleoside polyphosphates incorporated during a template-directed primer extension reaction.

In one aspect, the present invention provides a method of identifying a sequence of a plurality of template nucleic acids that includes the steps of: (a) providing a plurality of immobilized clonal populations of primed nucleic acid templates, each clonal population in contact with or proximate to an electronic sensing element; (b) exposing the plurality of immobilized clonal populations to a first type of nucleoside polyphosphate under conditions supporting a template directed incorporation of a nucleoside monophosphate portion of the first type of nucleoside polyphosphate; where the first type of nucleoside polyphosphate includes a polyphosphate chain of three or more phosphates and a terminal blocking group; where the incorporation reaction is carried out in the presence of a phosphatase enzyme and results in the cleavage of an alpha-beta phosphate bond and cleavage of at least one additional phosphate bond of the polyphosphate chain; (c) electrically monitoring each of the clonal populations with the electronic sensing elements to detect whether one or more incorporations of the first type of nucleoside polyphosphate occurs at that clonal population; and (d) repeating steps (b) and (c) with second, third and fourth types of nucleoside phosphates, where the repeating step (d) is conducted a number of times to thereby identify the sequence of the plurality of template nucleic acids.

In a further embodiment and in accordance with the above, the electronic sensing elements of use in methods of the present invention sense ionic changes, pH changes, temperature changes, or changes in magnetic field resulting from the cleavage of phosphate bonds.

In a still further embodiment and in accordance with any of the above, the electronic sensing element comprises a field effect transistor (FET) or an ion sensitive field effect transistor (ISFET).

In a still further embodiment and in accordance with any of the above, the clonal populations of primed nucleic acid templates are provided on beads or as separate regions on a substrate.

In a yet further embodiment and in accordance with any of the above, the polyphosphate chain comprises between 3 and 20 phosphates.

In a further embodiment and in accordance with any of the above, the polyphosphate chain comprises 3, 4, 5, 6, 7, 8, 9, 10, 11, or 12 phosphates.

In a further embodiment and in accordance with any of the above, the first, second, third, and fourth types of nucleoside polyphosphates each correspond to a nucleobase independently selected from A, G, C, or T.

In a further embodiment and in accordance with any of the above, the incorporation is carried out in the presence of a phosphatase enzyme for cleavage of the at least one additional phosphate bond.

In a further embodiment and in accordance with any of the above, the phosphatase enzyme comprises shrimp alkaline phosphatase or calf intestinal phosphatase.

In a still further embodiment and in accordance with any of the above, the terminal blocking group prevents phosphatase cleavage of the nucleoside polyphosphate prior to the incorporation reaction.

In a further embodiment and in accordance with any of the above, the terminal blocking group comprises a member selected from a methyl group, an amino hexyl group, a dye, an adduct, and a linker.

In a further embodiment and in accordance with any of the above, the number of immobilized clonal populations of primed nucleic acid templates is between 1,000 and 10 million or between 100,000 and 5 million.

In a further embodiment and in accordance with any of the above, cleavage of the at least one additional phosphate bond comprises cleavage of 2, 3, 4, 5, 6, 7, 8, 9, or 10 additional phosphate bonds.

In a further embodiment and in accordance with any of the above, the second, third and fourth types of nucleoside polyphosphates comprise a polyphosphate chain of four or more polyphosphates.

In a further embodiment and in accordance with any of the above, the electronic sensing elements sense changes in magnetic field caused by the cleavage of the phosphate bonds.

In one aspect, the present invention provides a method of identifying a sequence of a plurality of template nucleic acids, where the method includes the following steps: (a) providing a plurality of single-molecule polymerase-template complexes, each complex comprising a template nucleic acid, a polymerase enzyme and a primer; wherein each complex is associated with an electronic sensing element; (b) exposing the complexes to two or more types of nucleoside polyphosphates, wherein the two or more types of nucleoside polyphosphates each comprises a phosphate chain of three or more phosphates, and wherein each type of nucleoside polyphosphate has a different number of phosphates and a terminal blocking group; the exposing carried out under conditions supporting template dependent primer extension through multiple incorporation reactions, whereby the incorporation reactions extending the primer are carried out in the presence of a phosphatase enzyme resulting in the cleavage of an alpha-beta phosphate bond (by the polymerase) and at least one additional phosphate bond of the incorporated nucleoside polyphosphates; and (c) detecting the phosphate bond cleavages resulting from the incorporation reactions with the electronic sensing elements to identify the types of nucleoside polyphosphates incorporated in the incorporation reactions to thereby sequence the plurality of template nucleic acids.

In a further embodiment and in accordance with any of the above, the two or more types of nucleoside polyphosphates comprise four types of nucleoside polyphosphates corresponding to the nucleobases A, G, T, and C.

In a further embodiment and in accordance with any of the above, the electronic sensing elements of use in methods of the present invention sense ionic changes, pH changes, temperature changes, or changes in magnetic field resulting from the cleavage of phosphate bonds.

In a still further embodiment and in accordance with any of the above, the electronic sensing element comprises a field effect transistor (FET) or an ion sensitive field effect transistor (ISFET).

In a still further embodiment and in accordance with any of the above, the polymerase enzyme is immobilized on a substrate. In a further exemplary embodiment, the substrate is a zero mode waveguide.

In a further embodiment and in accordance with any of the above, the polyphosphates of the nucleoside polyphosphates comprise between 3 and 20 phosphates.

In a further embodiment and in accordance with any of the above, the polyphosphates of the nucleoside polyphosphates comprise 3, 4, 5, 6, 7, 8, 9, 10, 11, or 12 phosphates.

In a further embodiment and in accordance with any of the above, the phosphatase enzyme comprises shrimp alkaline phosphatase or calf intestinal phosphatase.

In a still further embodiment and in accordance with any of the above, the terminal blocking group prevents phosphatase cleavage of the nucleoside polyphosphate prior to the incorporation reaction.

In a further embodiment and in accordance with any of the above, the terminal blocking group comprises a member selected from a methyl group, an amino hexyl group, a dye, an adduct, and a linker.

In a further embodiment and in accordance with any of the above, the number of immobilized clonal populations of primed nucleic acid templates is between 1,000 and 10 million or between 100,000 and 5 million.

In a further embodiment and in accordance with any of the above, cleavage of the at least one additional phosphate bond comprises cleavage of 2, 3 4, 5, 6, 7, 8, 9, or 10 additional phosphate bonds.

In a further embodiment and in accordance with any of the above, the detecting step (c) comprises detecting signals generated by the phosphate bond cleavages, wherein one or more characteristics of the signals are used to identify the type of nucleoside polyphosphates incorporated in the incorporation reactions.

In one aspect, the present invention provides a method of identifying a sequence of a plurality of template nucleic acids, where the method includes the steps of: (a) providing a plurality of immobilized single-molecule primed nucleic acid templates, where each single molecule template is proximate to an electronic sensing element; (b) exposing the plurality of immobilized single molecules to a first type of nucleoside polyphosphate under conditions supporting a template directed incorporation of a nucleoside monophosphate portion of the first type of nucleoside polyphosphate and in the presence of a phosphatase enzyme, where the first type of nucleoside polyphosphate includes a polyphosphate chain of three or more phosphates and a terminal blocking group and where, upon incorporation, cleavage of the alpha-beta phosphate bond and cleavage of at least one additional phosphate bond of the polyphosphate chain occurs; (c) electrically monitoring each of the single molecule templates with the electronic sensing elements to detect whether one or more incorporations of the type of nucleoside polyphosphate occurs at that single-molecule template; (d) repeating steps (b) and (c) with second, third and fourth types of nucleoside phosphates, where the repeating step (d) is conducted a number of times to thereby identify the sequence of the plurality of template nucleic acids.

In one aspect, the present invention provides a method for increasing a signal from a template directed incorporation of a nucleoside monophosphate portion of a nucleoside polyphosphate, the method including the steps of: (a) providing a plurality of immobilized clonal populations of primed nucleic acid templates, each clonal population proximate to an electronic sensing element; (b) exposing the plurality of immobilized clonal populations to a first type of nucleoside polyphosphate under conditions supporting a template directed incorporation of a nucleoside monophosphate portion of the first type of nucleoside polyphosphate, where the first type of nucleoside polyphosphate comprises a polyphosphate chain of three or more phosphates and a terminal blocking group; and where, upon incorporation, cleavage of the alpha-beta phosphate bond and cleavage of at least one additional phosphate bond of the polyphosphate chain occurs, thereby generating a signal detectable by the electronic sensing elements; (c) electrically monitoring each of the clonal populations with the electronic sensing elements to detect whether one or more incorporations of the type of nucleoside polyphosphate occurs at that clonal population by detecting the signal generated by cleavage of the alpha-beta phosphate bond and the at least one additional phosphate bond; (d) repeating steps (b) and (c) with second, third and fourth types of nucleoside phosphates, wherein the repeating step (d) is conducted a number of times to identify the sequence of the plurality of template nucleic acids.

In one aspect the present invention provides a method for increasing a signal from a template directed incorporation of a nucleoside monophosphate portion of a nucleoside polyphosphate. In this aspect, the method includes the steps of: (a) providing a plurality of single-molecule polymerase-template complexes, each complex comprising a template nucleic acid, a polymerase enzyme and a primer, where each complex is associated with an electronic sensing element; (b) exposing the complexes to two or more types of nucleoside polyphosphates, where the two or more types of nucleoside polyphosphates each comprises a phosphate chain of three or more phosphates and a terminal blocking, and wherein each type of nucleoside polyphosphate has a different number of phosphates; the exposing carried out under conditions supporting template dependent primer extension through multiple incorporation reactions, whereby the incorporation reactions extending the primer are carried out in the presence of a phosphatase enzyme resulting in the cleavage of an alpha-beta phosphate bond and at least one additional phosphate bond of the incorporated nucleoside polyphosphates, thereby generating a signal detectable by the electronic sensing elements; and (c) detecting the signals from the phosphate bond cleavages resulting from the incorporation reactions with the electronic sensing elements to identify the types of nucleoside polyphosphates incorporated in the incorporation reactions to thereby sequence the plurality of template nucleic acids.

In one aspect, the present invention provides a method for identifying a sequence of a plurality of template nucleic acids that includes the steps of: (a) providing a plurality of immobilized clonal populations of nucleic acids, wherein each clonal population is proximate to an electronic sensing element; (b) exposing the plurality of immobilized clonal populations to a first type of nucleoside polyphosphate under conditions supporting a template directed incorporation of a nucleoside monophosphate portion of the first type of nucleoside polyphosphates into primers hybridized to the nucleic acids; wherein the first type of nucleoside polyphosphate comprises a polyphosphate chain of three or more phosphates and a terminal blocking group; and whereby, upon incorporation, cleavage of the alpha-beta phosphate bond and cleavage of at least one additional phosphate bond of the polyphosphate chain occurs, thereby releasing at least three hydrogen ions; (c) electrically monitoring each of the clonal populations with the electronic sensing elements to detect whether one or more incorporations of the first type of nucleoside polyphosphate occurs at that clonal population by detecting the released hydrogen ions at that clonal population; (d) repeating steps (b) and (c) with second, third and fourth types of nucleoside phosphates, wherein the repeating step (d) is conducted a number of times to thereby identify the sequence of the plurality of template nucleic acids.

In a further aspect, the present invention provides a method for identifying a sequence of a plurality of template nucleic acids that includes the steps of: (a) providing a plurality of immobilized clonal populations of primed nucleic acid templates, each clonal population proximate to an electronic sensing element; (b) exposing the plurality of immobilized clonal populations to a first type of nucleoside polyphosphate under conditions supporting a template directed incorporation of a nucleoside monophosphate portion of the first type of nucleoside polyphosphate; wherein the first type of nucleoside polyphosphate comprises a polyphosphate chain of three or more phosphates; and whereby, upon incorporation, cleavage of the alpha-beta phosphate bond and cleavage of at least one additional phosphate bond of the polyphosphate chain occurs, thereby generating a byproduct detectable by the electronic sensing element; (c) electrically monitoring each of the clonal populations with the electronic sensing elements to detect whether one or more incorporations of the type of nucleoside polyphosphate occurs at that clonal population by detecting the byproduct generated by the cleavage of the phosphate bonds; (d) repeating steps (b) and (c) with second, third and fourth types of nucleoside phosphates, wherein the repeating step (d) is conducted a number of times to thereby identify the sequence of the plurality of template nucleic acids.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, phage display, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

By “nucleic acid” or “oligonucleotide” or grammatical equivalents herein means at least two nucleotides covalently linked together. A nucleic acid of the present invention will generally contain phosphodiester bonds, although in some cases, nucleic acid analogs are included that may have alternate backbones, comprising, for example, phosphoramide, phosphorothioate, phosphorodithioate, and peptide nucleic acid backbones and linkages. Other analog nucleic acids include those with positive backbones; non-ionic backbones, and non-ribose backbones, including those described in U.S. Pat. Nos. 5,235,033 and 5,034,506. The template nucleic acid may also have other modifications, such as the inclusion of heteroatoms, the attachment of labels, such as dyes, or substitution with functional groups which will still allow for base pairing and for recognition by the enzyme.

As used herein, a “substantially identical” nucleic acid is one that has at least 80%, 85%, 90%, 95%, 96%, 97%, 98% or 99% sequence identity to a reference nucleic acid sequence. The length of comparison is preferably the full length of the nucleic acid, but is generally at least 20 nucleotides, 30 nucleotides, 40 nucleotides, 50 nucleotides, 75 nucleotides, 100 nucleotides, 125 nucleotides, or more.

### I. Overview

The present invention is directed to methods, devices, compositions and systems for obtaining sequence data from nucleic acid templates. In some aspects, the methods generally comprise stepwise electronic sequence of a plurality of template nucleic acids. In other aspects, the methods comprise real-time single-molecule sequencing. In general, the methods involve detecting a signal that is associated with the cleavage of polyphosphate chains released from nucleoside polyphosphates incorporated during a template-directed primer extension reaction.

A signal “associated with” the cleavage of polyphosphate chains as used herein refers to a signal whose intensity or characteristics are affected by the number of ions, such as hydrogen ions, that are released when a polyphosphate chain is cleaved. As will be discussed in further detail herein, such signals include without limitation measurements of pH, measurements of concentration of phosphate ion, measurements of changes in temperature, measurements of changes in magnetic fields, and measurements of conformational changes of phosphate binding proteins. As will be appreciated, these measurements can include measurements of intensity as well as kinetics.

In some aspects, methods of the present invention include methods of identifying a sequence of a plurality of template nucleic acids in which a plurality of immobilized clonal populations of primed nucleic acids are provided such that each clonal population is in contact with or proximate to an electronic sensing element. The electronic sensing element is associated with the clonal population such that the chemical reactions that occur within the clonal populations are sensed by the electronic element. In some cases the nucleic acids or polymerase-nucleic acid complexes are immobilized on the electronic sensing element. In other cases the nucleic acid templates are close enough (proximate) to the sensing element that ionic or electromagnetic changes that occur upon incorporation of the nucleoside monophosphate portion of a nucleoside polyphosphate are detected by the electronic sensing elements (also referred to herein as “sensing elements”). In some cases, the template nucleic acids are on particles or beads that are close enough to the sensing elements to allow detection of the incorporation reactions. The sensing elements can be within small chambers into which the beads or particles comprising the template nucleic acids are delivered. The electronic sensing elements of use in the present invention may include without limitation elements that sense ionic changes or pH changes, elements that sense temperature changes, elements that sense changes in magnetic field, a field effect transistor, and ion sensitive field effect transistors. In further aspects, the methods of the present invention include exposing the plurality of immobilized clonal populations to a first type of nucleoside polyphosphate that comprises a polyphosphate chain of three or more phosphates. These immobilized clonal populations are exposed to the first type of nucleoside polyphosphates under conditions supporting a template directed incorporation of the nucleoside monophosphate portion of the first type of nucleoside polyphosphates into a growing chain, typically extending from a primer. Upon such an incorporation (which, as will be appreciated, occurs if the first type of nucleoside polyphosphate comprises a nucleobase complementary to a base of the template nucleic acid), the alpha-beta phosphate bond of the first type of nucleoside polyphosphate is cleaved by a polymerase enzyme that adds the nucleoside monophosphate to the growing chain. In addition to the cleavage of the alpha-beta phosphate bond, in the current method, at least one other phosphate bond is cleaved, generally by an enzyme such as a phosphatase, although chemical cleavage reactions are also contemplated. The incorporation of the first type of nucleoside polyphosphate results in the release of a polyphosphate chain and the cleavage of at least one additional phosphate bond of that polyphosphate chain. Thus, the incorporation of the first type of nucleoside polyphosphate results in the cleavage of at least two phosphate bonds per incorporation event. By cleaving two or more phosphate bonds in the polyphosphate chain, one obtains an amplification of the signal at the electronic detector over what would be detected with the cleavage of only one bond. In some aspects substantially all of the phosphate bonds in the chain are cleaved. For example where a tetraphosphate is used, typically three phosphate bonds will be cleaved (e.g. two by the phosphatase and one by the polymerase). That is, the polymerase cleaves at the alpha-beta bond to release a triphosphate which is in turn cleaved into three individual phosphates by cleavage of the two remaining phosphate bonds. Analogously, where there is pentaphosphate, the cleavage of the alpha-beta bond by the polymerase results in the release of a tetraphosphate which is cleaved, for example by a phosphatase into four phosphate ions by the cleavage of the remaining three phosphate bonds. This approach can be extended as described herein to a hexaphosphate, heptaphosphate, octaphosphate, nonaphosphate, decaphosphate, etc. In further aspects of the invention, each of the clonal populations is electrically monitored with the electronic sensing elements to detect whether one or more incorporations of the first type of nucleoside polyphosphate occurs at that clonal population, thereby identifying a nucleotide of the template nucleic acid at that clonal population. In still further aspects, the exposing and detecting steps are repeated with a second, third and fourth type of nucleoside polyphosphates enough times to identify the sequence of the plurality of template nucleic acids. In yet further aspects, the nucleoside polyphosphates further comprise terminal blocking groups to prevent cleavage of the polyphosphate chain prior to the incorporation event.

In aspects of the invention involving single molecule sequencing, methods of the invention include providing a plurality of single-molecule polymerase-template complexes, where each complex includes a template nucleic acid, a polymerase enzyme and a primer. Each complex is also associated with an electronic sensing element. As with the stepwise sequencing method discussed above, that electronic sensing element may include without limitation an element that senses ionic changes or pH changes, an element that senses temperature changes, an element that senses changes in magnetic field, a field effect transistor, and an ion sensitive field effect transistor. In further aspects, the single molecule sequencing methods of the invention include a step of exposing the complexes to two or more types of nucleoside polyphosphates, where the two or more types of nucleoside polyphosphates each comprises a phosphate chain of three or more phosphates. In addition, each type of nucleoside polyphosphate has a different number of phosphates. The exposing step is carried out under conditions supporting template dependent primer extension through multiple incorporation reactions. Each of these multiple incorporation reactions results in the cleavage of an alpha-beta phosphate bond and at least one additional phosphate bond of the polyphosphate chain of the incorporated nucleoside polyphosphates. Thus, as with the stepwise sequencing methods discussed above, the real-time single molecule sequencing methods of the present invention result in the cleavage of multiple phosphate bonds per incorporation event—as a result, any signal associated with the cleavage of the multiple phosphate bonds is larger than would be possible for incorporation events in which only a single phosphate bond is cleaved. The cleavage of the phosphate bonds other than the alpha-beta phosphate bond is generally accomplished by an enzyme such as a phosphatase, although, as is discussed above and in further detail herein, chemical phosphate bond cleavage reactions are also contemplated. As discussed above for the stepwise sequencing methods, the nucleoside polyphosphates will in general include terminal blocking groups to prevent cleavage of the polyphosphate chain prior to the incorporation event.

The phosphate bond cleavages in both the stepwise and single molecule methods are detected by the electronic sensing elements identify the types of nucleoside polyphosphates incorporated in the incorporation reactions, and thereby sequence the plurality of template nucleic acids. This detecting step includes using one or more characteristics of the signals generated by the phosphate bond cleavages to identify the type of nucleoside polyphosphates incorporated in the incorporation reactions.

The above aspects and further exemplary embodiments are described in further detail in the following discussion.

### II. Compositions

The present invention provides compositions and methods for obtaining sequence data from nucleic acid templates. In some aspects, the methods generally comprise stepwise electronic sequence of a plurality of template nucleic acids. In other aspects, the methods comprise real-time single-molecule sequencing. The compositions discussed in this section can be used in any of the methods described in further detail herein.

II.A. Nucleotide Analogs

Any of the methods described herein utilize nucleoside polyphosphates (also referred to herein as “nucleotide analogs” and “nucleoside polyphosphate analogs”) that have a relatively high number of phosphate groups. In exemplary embodiments, nucleotide analogs of use in methods of the invention have at least 4, 5, 6, 7, 8, 9, 10, 11, 12, 13, 14, 15, 16, 17, 18, 19, 20, 21, 22, 23, 24, 25, 26, 27, 28, 29, 30 phosphate groups. In further exemplary embodiments, nucleotide analogs of use in methods of the invention have about 4, 5, 6, 7, 8, 9, 10, 11, 12, 13, 14, 15, 16, 17, 18, 19, 20, 21, 22, 23, 24, 25, 26, 27, 28, 29, 30 phosphate groups. In still further exemplary embodiments, nucleotide analogs of the invention have from about 4-60, 5-55, 6-50, 7-45, 8-40, 9-35, 10-30, 11-25, 12-20, 13-15, 4-20, 4-12, 5-19, 6-18, 7-17, 8-16, 9-15, 10-14, 11-13 phosphate groups. In still further embodiments, the methods of the invention described herein do not utilize nucleotide triphosphates (i.e., nucleoside polyphosphates with three phosphate groups).

In further embodiments and in accordance with any of the above, the nucleotide analogs of use in the present invention include 4 or more phosphate groups as discussed above and in addition include a terminal protecting group (also referred to herein as a “terminal blocking group”) to protect the nucleotide analog from degradation until the nucleotide analog is incorporated and the polyphosphate chain is released, for example in one or more of the template-directed polymerization reactions in the stepwise and single molecule sequencing reactions discussed herein. The protecting group will in general be on the terminal phosphate of the polyphosphate chain of the nucleotide analog and can be any type of protecting group that prevent a hydrolysis reaction, such as a reaction by a phosphatase. In some embodiments, the nucleoside polyphosphate is protected by another nucleoside of the same base (e.g., a symmetric dinucleoside polyphosphate). In one non-limiting embodiment, the protecting group includes any group that takes the place of one or more of the oxygen atoms of the terminal phosphate group to prevent degradation. In further exemplary embodiments, the protecting group comprises a linker, an alkyl group (including without limitation a methyl, ethyl, propyl or butyl group), a dye, any other adduct (including without limitation a fluorophore, a carbohydrate, and an aromatic group) that is attached either to the P or an O in the terminal phosphate. In embodiments in which the protecting group is a linker, the linker can be any molecular structure, including without limitation organic linkers such as alkane or alkene linkers of from about C2 to about C20, or longer, polyethyleneglycol (PEG) linkers, aryl, heterocyclic, saturated or unsaturated aliphatic structures comprised of single or connected rings, amino acid linkers, peptide linkers, nucleic acid linkers, PNA, LNAs, or the like or phosphate or phosphonate group containing linkers. In some embodiments, alkyl, e.g., alkane, alkene, alkyne alkoxy or alkenyl, or ethylene glycol linkers are used. Some examples of linkers are described in Published U.S. Patent Application No. 2004/0241716, which is incorporated herein by reference in its entirety for all purposes and in particular for all teachings related to linkers. The protecting groups may in further embodiments be alkyl, aryl, or ester linkers. The protecting groups may also be amino-alkyl linkers, e.g., amino-hexyl linkers. In some cases, the linkers can be rigid linkers such as disclosed in U.S. patent application Ser. No. 12/403,090, which is incorporated herein by reference in its entirety for all purposes and in particular for all teachings related to linkers.

As will be discussed in further detail herein, methods of the invention utilize one or more types of nucleotide analogs. In some embodiments, each of the different types of nucleotides will have a different number of phosphate groups in the polyphosphate chain, such that each type may be identified from each other type upon incorporation. For example, each of the different types of nucleotide analogs may each correspond to a nucleobase independently selected from A, G, C, or T (or to one or more modified nucleobases), and each type may be distinguished from the other types based on characteristics such as the signal generated when the nucleotide analog is incorporated during a polymerase reaction. For example, each type of nucleotide analog can in some embodiments have a different number of phosphate groups in the polyphosphate chain, such that, upon incorporation of a particular nucleotide analog type during a polymerization reaction, the signal associated with the resultant cleavage of the phosphate bonds of the polyphosphate chain will identify the incorporated nucleotide analog as having a nucleobase A, C, G, or T. In further embodiments, sequencing reactions discussed herein may utilize 1, 2, 3, 4, 5, 6, 7, 8, 9, 10, 11, 12 or more different types of nucleotide analogs, and in further exemplary embodiments each of the different types of nucleotide analogs has a different number of phosphate groups in their polyphosphate chains.

In addition to the naturally occurring “nucleobases,” adenine, cytosine, guanine and thymine (A, C, G, T), nucleic acid components of the compounds of the invention optionally include modified bases. These components can also include modified sugars. For example, the nucleic acid can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, nitroindole, and 2,6-diaminopurine. The dye of the invention or another probe component can be attached to the modified base.

In further embodiments, the nucleotide analogs of the present invention may further include labels, such as fluorescent labeling groups. These labeling groups may also be such that the different types of nucleotide analogs may be distinguished from one another. In such embodiments, typically, each of the different types of nucleotide analogs will be labeled with a detectably different fluorescent labeling group, e.g., that possesses a detectably distinct fluorescent emission and/or excitation spectrum, such that it may be identified and distinguished from different nucleotides upon incorporation. For example, each of the different types of nucleotides, e.g., A, T, G and C, will be labeled with a fluorophore having a different emission spectrum. For certain embodiments, the nucleotide may include a fluorescent labeling group coupled to a portion of the nucleotide that is incorporated into the nascent nucleic acid strand being produced during synthesis, e.g., the nucleobase or sugar moiety. Nucleotide compositions having fluorophores coupled to these portions have been previously described (See, e.g., U.S. Pat. Nos. 5,476,928 and 4,711,955 to Ward et al.). As a result of the label group being coupled to the base or sugar portion of the nucleotide, upon incorporation, the nascent strand will include the labeling group. This labeling group may then remain or be removed, e.g., through the use of cleavable linkages joining the label to the nucleotide (See, e.g., U.S. Pat. No. 7,057,026). A variety of different fluorophore types, including both organic and inorganic fluorescent materials, have been described for biological applications and are likewise applicable in the instant invention.

In further embodiments, nucleotide analogs of the present invention may include nucleoside polyphosphates having the structure:

B-S-P-G,

wherein B is a natural or non-natural nucleobase, S is selected from a sugar moiety, an acyclic moiety or a carbocyclic moiety, P is a modified or unmodified polyphosphate, and G is a protecting group.

The base moiety, B, incorporated into the nucleotide analogs of the invention is generally selected from any of the natural or non-natural nucleobases or nucleobase analogs, including, e.g., purine or pyrimidine bases that are routinely found in nucleic acids and nucleic acid analogs, including adenine, thymine, guanine, cytidine, uracil, and in some cases, inosine. For purposes of the present description, nucleotides and nucleotide analogs are generally referred to based upon their relative analogy to naturally occurring nucleotides. As such, an analog that operates, functionally, like adenosine triphosphate, may be generally referred to herein by the shorthand letter A. Likewise, the standard abbreviations of T, G, C, U and I, may be used in referring to analogs of naturally occurring nucleosides and nucleotides typically abbreviated in the same fashion. In some cases, a base may function in a more universal fashion, e.g., functioning like any of the purine bases in being able to hybridize with any pyrimidine base, or vice versa. The base moieties used in the present invention may include the conventional bases described herein or they may include such bases substituted at one or more side groups, or other fluorescent bases or base analogs, such as 1, N6 ethenoadenosine or pyrrolo C, in which an additional ring structure renders the B group neither a purine nor a pyrimidine. For example, in certain cases, it may be desirable to substitute one or more side groups of the base moiety with a labeling group or a component of a labeling group, such as one of a donor or acceptor fluorophore, or other labeling group. Examples of labeled nucleobases and processes for labeling such groups are described in, e.g., U.S. Pat. Nos. 5,328,824 and 5,476,928, each of which is incorporated herein by reference in its entirety for all purposes and in particular for all teachings related to nucleobases and labeling nucleobases.

In the nucleotide analogs of the invention, the S group is generally a sugar moiety that provides a suitable backbone for a synthesizing nucleic acid strand. In it most preferred aspect, the sugar moiety is selected from a D-ribosyl, 2′ or 3′ D-deoxyribosyl, 2′,3′-D-dideoxyribosyl, 2′,3′-D-didehydrodideoxyribosyl, 2′ or 3′ alkoxyribosyl, 2′ or 3′ aminoribosyl, 2′ or 3′ mercaptoribosyl, 2′ or 3′ alkothioribosyl, acyclic, carbocyclic or other modified sugar moieties. A variety of carbocyclic or acyclic moieties may be incorporated as the “S” group in place of a sugar moiety, including, e.g., those described in published U.S. Patent Application No. 2003/0124576, incorporated herein by reference in its entirety for all purposes and in particular for all teachings related to sugar moieties of nucleotides and nucleotide analogs.

The P groups in the nucleotides of the invention are modified or unmodified polyphosphate groups. As discussed above, the number of phosphates in the polyphosphate can have 4, 5, 6, 7, 8, 9, 10, 11, 12, 13, 14, 15, 16, 17, 18, 19, 20, 21, 22, 23, 24, 25, 26, 27, 28, 29, 30 phosphate groups or more modified or unmodified phosphates. The unmodified phosphates have linearly linked—O—P(O)₂— units, for example a tetraphosphate, pentaphosphate, hexaphosphate, heptaphosphate, or octaphosphate. The P groups also include modified polyphosphates, for example by virtue of the inclusion of one or more phosphonate groups, effectively substituting a non-ester linkage in the phosphorous containing chain of the analog, with a more stable linkage. Examples of preferred linkages include, e.g., CH₂, methylene derivatives (e.g., substituted independently at one or more hydrogens with F, Cl, OH, NH₂, alkyl, alkenyl, alkynyl, etc.), CCl₂, CF₂, NH, S, CH₂CH₂, C(OH)(CH₃), C(NH₂)[(CH₂)₆CH₃], CH(NHR) (R is H or alkyl, alkenyl, alkynyl, aryl, C(OH)[(CH₂)ₙNH₂] (n is 2 or 3), and CNH₂. In particularly preferred aspects, methylene, amide or their derivatives are used as the linkages.

Other P groups of the invention have phosphate or modified phosphates in which one or more non-bridging oxygen is substituted, for example with S, or BH₃. In one aspect of the invention, one or more, two or more, three or more, or four or more non-bridging oxygen atoms in the P group has an S substituted for an O. The substitution of, sulfur atoms for oxygen can change the polymerase reaction kinetics such that a system having two slow steps can be selected. While not being bound by theory, it is believed that the properties of the nucleotide, such as the metal chelation properties, electronegativity, or steric properties are the nucleotide can be altered by the substitution of non-bridging oxygen for sulfur in P. In some cases, it is believed that the substitution of two or more non-bridging oxygen atoms with sulfur can affect the metal chelation properties so as to lead to a change in the kinetics of incorporation, which can be used to modulate the signals generated from the incorporation events discussed herein.

Suitable nucleotide analogs include analogs in which sulfur is substituted for one of the non-bridging oxygens. In some embodiments, the single sulfur substitution is made such that substantially only one stereoisomer is present. The nucleotide can have multiple phosphates in which one or more of the phosphates has a non-bridging sulfur in place of oxygen. The substituted phosphate in the nucleotide can be the R or the S stereoisomer.

G generally refers to a protecting group that is coupled to the terminal phosphorus atom via the R₄ (or R₁₀ or R₁₂) group. As discussed above, the protecting groups employed in the analogs of the invention may comprise any of a variety of molecules, including a linker, an alkyl group (including without limitation a methyl, ethyl, propyl or butyl group), any other adduct (including without limitation a fluorophore, a carbohydrate, and an aromatic group) or a label e.g., optical labels, e.g., labels that impart a detectable optical property to the analog, electrochemical labels, e.g., labels that impart a detectable electrical or electrochemical property to the analog, physical labels, e.g., labels that impart a different physical or spatial property to the analog, e.g., a mass tag or molecular volume tag. In some cases individual labels or combinations may be used that impart more than one of the aforementioned properties to the nucleotide analogs of the invention.

The protecting group may be directly coupled to the terminal phosphorus atom of the analog structure, in alternative aspects, it may additionally include a linker molecule to provide the coupling through, e.g., an alkylphosphonate linkage. A wide variety of linkers and linker chemistries are known in the art of synthetic chemistry may be employed in coupling the labeling group to the analogs of the invention. For example, such linkers may include organic linkers such as alkane or alkene linkers of from about C2 to about C20, or longer, polyethyleneglycol (PEG) linkers, aryl, heterocyclic, saturated or unsaturated aliphatic structures comprised of single or connected rings, amino acid linkers, peptide linkers, nucleic acid linkers, PNA, LNAs, or the like or phosphate or phosphonate group containing linkers. In preferred aspects, alkyl, e.g., alkane, alkene, alkyne alkoxy or alkenyl, or ethylene glycol linkers are used. Some examples of linkers are described in Published U.S. Patent Application No. 2004/0241716, which is incorporated herein by reference in its entirety for all purposes. Additionally, such linkers may be selectively cleavable linkers, e.g., photo- or chemically cleavable linkers or the like. The linkers can be alkyl, aryl, or ester linkers. The linkers can be, amino-alkyl linkers, e.g., amino-hexyl linkers. In some cases, the linkers can be rigid linkers such as disclosed in U.S. patent application Ser. No. 12/403,090.

The B, S, P, and G groups can be connected directly, or can be connected using an linking unit such as an —O—, —S—, —NH—, or —CH₂— unit.

II.B. Template Nucleic Acids

The present invention provides compositions and methods for identifying the sequences of template nucleic acids (also referred to herein as “template sequences”). In general, the template nucleic acid is the molecule for which the complimentary sequence is synthesized in the polymerase reaction. In some cases, the template nucleic acid is linear, in some cases, the template nucleic acid is circular. The template nucleic acid can be DNA, RNA, or can be a non-natural RNA analog or DNA analog. Any template nucleic acid that is suitable for replication by a polymerase enzyme can be used herein.

The template sequence may be provided in any of a number of different format types depending upon the desired application. For example, in some cases, the template sequence may be a linear single or double stranded nucleic acid sequence. In still other embodiments, the template may be provided as a circular or functionally circular construct that allows redundant processing of the same nucleic acid sequence by the synthesis complex. Use of such circular constructs has been described in, e.g., U.S. Pat. No. 7,315,019 and U.S. patent application Ser. No. 12/220,674, filed Jul. 25, 2008, alternate functional circular constructs are also described in US Pat. App. Pub. No. 20090298075 the full disclosures of each of which are incorporated herein by reference in their entirety for all purposes and in particular for all teachings related to template nucleic acid constructs.

Briefly, such alternate constructs include template sequences that possess a central double stranded portion that is linked at each end by an appropriate linking oligonucleotide, such as a hairpin loop segment. Such structures not only provide the ability to repeatedly replicate a single molecule (and thus sequence that molecule), but also provide for additional redundancy by replicating both the sense and antisense portions of the double stranded portion. In the context of sequencing applications, such redundant sequencing provides great advantages in terms of sequence accuracy.

In further embodiments, genomic DNA is obtained from a sample and fragmented for use in methods of the invention. The fragments may be single or double stranded and may further be modified in accordance with any methods known in the art and described herein. Template nucleic acids may be generated by fragmenting source nucleic acids, such as genomic DNA, using any method known in the art. In one embodiment, shear forces during lysis and extraction of genomic DNA generate fragments in a desired range. Also encompassed by the invention are methods of fragmentation utilizing restriction endonucleases.

As will be appreciated, the sample from which DNA is obtained may comprise any number of things, including, but not limited to, bodily fluids (including, but not limited to, blood, urine, serum, lymph, saliva, anal and vaginal secretions, perspiration and semen) and cells of virtually any organism, with mammalian samples being preferred and human samples being particularly preferred; environmental samples (including, but not limited to, air, agricultural, water and soil samples); biological warfare agent samples; research samples (i.e. in the case of nucleic acids, the sample may be the products of an amplification reaction, including both target and signal amplification, such as PCR amplification reactions; purified samples, such as purified genomic DNA, RNA preparations, raw samples (bacteria, virus, genomic DNA, etc.); as will be appreciated by those in the art, virtually any experimental manipulation may have been done on the samples.

Target nucleic acids may be generated from a source nucleic acid, such as genomic DNA, by fragmentation to produce fragments of a specific size. The target nucleic acids can be, for example, from about 10 to about 50,000 nucleotides in length, or from about 10 to about 20,000 nucleotides in length. In one embodiment, the fragments are 50 to 600 nucleotides in length. In another embodiment, the fragments are 300 to 600 or 200 to 2000 nucleotides in length. In yet another embodiment, the fragments are 10-100, 50-100, 50-300, 100-200, 200-300, 50-400, 100-400, 200-400, 400-500, 400-600, 500-600, 50-1000, 100-1000, 200-1000, 300-1000, 400-1000, 500-1000, 600-1000, 700-1000, 700-900, 700-800, 800-1000, 900-1000, 1500-2000, 1750-2000, and 50-2000 nucleotides in length.

II. C. Polymerases

The methods of the present invention utilize polymerase enzymes (also referred to herein as “polymerases”). Any suitable polymerase enzyme can be used in the systems and methods of the invention. Suitable polymerases include DNA dependent DNA polymerases, DNA dependent RNA polymerases, RNA dependent DNA polymerases (reverse transcriptases), and RNA dependent RNA polymerases.

DNA polymerases are sometimes classified into six main groups based upon various phylogenetic relationships, e.g., with E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of recent nomenclature, see, e.g., Burgers et al. (2001) “Eukaryotic DNA polymerases: proposal for a revised nomenclature” J Biol Chem. 276(47):43487-90. For a review of polymerases, see, e.g., Hübscher et al. (2002) “Eukaryotic DNA Polymerases” Annual Review of Biochemistry Vol. 71: 133-163; Alba (2001) “Protein Family Review: Replicative DNA Polymerases” Genome Biology 2(1):reviews 3002.1-3002.4; and Steitz (1999) “DNA polymerases: structural diversity and common mechanisms” J Biol Chem 274:17395-17398. The basic mechanisms of action for many polymerases have been determined. The sequences of literally hundreds of polymerases are publicly available, and the crystal structures for many of these have been determined, or can be inferred based upon similarity to solved crystal structures of homologous polymerases. For example, the crystal structure of φ29, a preferred type of parental enzyme to be modified according to the invention, is available.

In addition to wild-type polymerases, chimeric polymerases made from a mosaic of different sources can be used. For example, φ29 polymerases made by taking sequences from more than one parental polymerase into account can be used as a starting point for mutation to produce the polymerases of the invention. Chimeras can be produced, e.g., using consideration of similarity regions between the polymerases to define consensus sequences that are used in the chimera, or using gene shuffling technologies in which multiple φ29-related polymerases are randomly or semi-randomly shuffled via available gene shuffling techniques (e.g., via “family gene shuffling”; see Crameri et al. (1998) “DNA shuffling of a family of genes from diverse species accelerates directed evolution” Nature 391:288-291; Clackson et al. (1991) “Making antibody fragments using phage display libraries” Nature 352:624-628; Gibbs et al. (2001) “Degenerate oligonucleotide gene shuffling (DOGS): a method for enhancing the frequency of recombination with family shuffling” Gene 271:13-20; and Hiraga and Arnold (2003) “General method for sequence-independent site-directed chimeragenesis: J. Mol. Biol. 330:287-296). In these methods, the recombination points can be predetermined such that the gene fragments assemble in the correct order. However, the combinations, e.g., chimeras, can be formed at random. For example, using methods described in Clarkson et al., five gene chimeras, e.g., comprising segments of a Phi29 polymerase, a PZA polymerase, an M2 polymerase, a B103 polymerase, and a GA-1 polymerase, can be generated. Appropriate mutations to improve branching fraction, increase closed complex stability, or alter reaction rate constants can be introduced into the chimeras.

Available DNA polymerase enzymes have also been modified in any of a variety of ways, e.g., to reduce or eliminate exonuclease activities (many native DNA polymerases have a proof-reading exonuclease function that interferes with, e.g., sequencing applications), to simplify production by making protease digested enzyme fragments such as the Klenow fragment recombinant, etc. As noted, polymerases have also been modified to confer improvements in specificity, processivity, and improved retention time of labeled nucleotides in polymerase-DNA-nucleotide complexes (e.g., WO 2007/076057 POLYMERASES FOR NUCLEOTIDE ANALOGUE INCORPORATION by Hanzel et al. and WO 2008/051530 POLYMERASE ENZYMES AND REAGENTS FOR ENHANCED NUCLEIC ACID SEQUENCING by Rank et al.), to alter branch fraction and translocation (e.g., U.S. patent application Ser. No. 12/584,481 filed Sep. 4, 2009, by Pranav Patel et al. entitled “ENGINEERING POLYMERASES AND REACTION CONDITIONS FOR MODIFIED INCORPORATION PROPERTIES”), to increase photostability (e.g., U.S. patent application Ser. No. 12/384,110 filed Mar. 30, 2009, by Keith Bjornson et al. entitled “Enzymes Resistant to Photodamage”), and to improve surface-immobilized enzyme activities (e.g., WO 2007/075987 ACTIVE SURFACE COUPLED POLYMERASES by Hanzel et al. and WO 2007/076057 PROTEIN ENGINEERING STRATEGIES TO OPTIMIZE ACTIVITY OF SURFACE ATTACHED PROTEINS by Hanzel et al.). Any of these available polymerases can be modified in accordance with the methods known in the art to decrease branching fraction formation, improve stability of the closed polymerase-DNA complex, and/or alter reaction rate constants. In some cases, the polymerase is modified in order to more effectively incorporate the nucleotide analogs of the invention, e.g. analogs having four or more phosphates in their polyphosphate chain, and/or nucleotide analogs having terminal groups to prevent phosphate cleavage by phosphatase enzymes. Enzymes mutated to more readily accept nucleotide analogs having such properties are described, for example in the applications described above and in US 20120034602—Recombinant Polymerases for Improved Single Molecule Sequencing; US 20100093555—Enzymes Resistant to Photodamage; US 20110189659—Generation of Modified Polymerases for Improved Accuracy in Single Molecule Sequencing; US 20100112645—Generation of Modified Polymerases for Improved Accuracy in Single Molecule Sequencing; US 2008/0108082—Polymerase enzymes and reagents for enhanced nucleic acid sequencing; and US 20110059505—Polymerases for Nucleotide Analogue Incorporation which are incorporated herein by reference in their entirety for all purposes.

Many polymerases that are suitable for modification are available, e.g., for use in sequencing, labeling and amplification technologies. For example, human DNA Polymerase Beta is available from R&D systems. DNA polymerase I is available from Epicenter, GE Health Care, Invitrogen, New England Biolabs, Promega, Roche Applied Science, Sigma Aldrich and many others. The Klenow fragment of DNA Polymerase I is available in both recombinant and protease digested versions, from, e.g., Ambion, Chimerx, eEnzyme LLC, GE Health Care, Invitrogen, New England Biolabs, Promega, Roche Applied Science, Sigma Aldrich and many others. φ29 DNA polymerase is available from e.g., Epicentre. Poly A polymerase, reverse transcriptase, Sequenase, SP6 DNA polymerase, T4 DNA polymerase, T7 DNA polymerase, and a variety of thermostable DNA polymerases (Taq, hot start, titanium Taq, etc.) are available from a variety of these and other sources. Recent commercial DNA polymerases include Phusion™ High-Fidelity DNA Polymerase, available from New England Biolabs; GoTaq® Flexi DNA Polymerase, available from Promega; RepliPHI™ φ29 DNA Polymerase, available from Epicentre Biotechnologies; PfuUltra™ Hotstart DNA Polymerase, available from Stratagene; KOD HiFi DNA Polymerase, available from Novagen; and many others. Biocompare(dot)com provides comparisons of many different commercially available polymerases.

DNA polymerases that are preferred substrates for mutation to decrease branching fraction, increase closed complex stability, or alter reaction rate constants include Taq polymerases, exonuclease deficient Taq polymerases, E. coli DNA Polymerase 1, Klenow fragment, reverse transcriptases, φ29-related polymerases including wild type φ29 polymerase and derivatives of such polymerases such as exonuclease deficient forms, T7 DNA polymerase, T5 DNA polymerase, an RB69 polymerase, etc.

In one aspect, the polymerase of use in the methods described herein is a modified φ29-type DNA polymerase. For example, the modified recombinant DNA polymerase can be homologous to a wild-type or exonuclease deficient φ29 DNA polymerase, e.g., as described in U.S. Pat. Nos. 5,001,050, 5,198,543, or 5,576,204. Alternately, the modified recombinant DNA polymerase can be homologous to other φ29-type DNA polymerases, such as B103, GA-1, PZA, φ15, BS32, M2Y, Nf, G1, Cp-1, PRD1, PZE, SFS, Cp-5, Cp-7, PR4, PRS, PR722, L17, φ21, or the like. For nomenclature, see also, Meijer et al. (2001) “φ29 Family of Phages” Microbiology and Molecular Biology Reviews, 65(2):261-287. Suitable polymerases are described, for example, in U.S. patent application Ser. No. 12/924,701, filed Sep. 30, 2010; and Ser. No. 12/384,112, filed Mar. 30, 2009.

In further embodiments, the polymerase enzyme used in the methods of the invention includes RNA dependent DNA polymerases or reverse transcriptases. Suitable reverse transcriptase enzymes include HIV-1, M-MLV, AMV, and Telomere Reverse Transcriptase. Reverse transcriptases also allow for the direct sequencing of RNA substrates such as messenger RNA, transfer RNA, non-coding RNA, ribosomal RNA, micro RNA or catalytic RNA.

The polymerase enzymes of the invention generally require a primer, which is usually a short oligonucleotide that is complementary to a portion of the template nucleic acid. The primers of the invention can comprise naturally occurring RNA or DNA oligonucleotides. The primers of the invention may also be synthetic analogs. The primers may have alternative backbones as described above for the nucleic acids of the invention. The primer may also have other modifications, such as the inclusion of heteroatoms, the attachment of labels, such as dyes, or substitution with functional groups which will still allow for base pairing and for recognition by the enzyme. Primers can select tighter binding primer sequences, e.g., GC rich sequences, as well as employ primers that include within their structure non-natural nucleotides or nucleotide analogs, e.g., peptide nucleic acids (PNAs) or locked nucleic acids (LNAs), that can demonstrate higher affinity pairing with the template. The primer can also be selected to influence the kinetics of the polymerase reaction.

II.D. Supports and Substrates

Substrates of use in particular sequencing methods of the invention are discussed in further detail herein, and as will be appreciated, any of the substrates discussed herein can be used in any combination for any embodiment of sequencing reaction. In exemplary embodiments, methods of sequencing of the invention utilize substrates that include one or more reaction chambers arranged in the form of an array on an inert substrate material, also referred to herein as a “solid support”, that allows for combination of the reactants in a sequencing reaction in a defined space and for detection of the sequencing reaction event. A reaction chamber can be a localized area on the substrate material that facilitates interaction of reactants, e.g., in a nucleic acid sequencing reaction. As discussed more fully below, the sequencing reactions contemplated by the invention can in some embodiments occur on numerous individual nucleic acid samples in tandem, in particular simultaneously sequencing numerous nucleic acid samples derived from genomic and chromosomal DNA. The apparatus of the invention can therefore include an array having a sufficient number of reaction chambers to carry out such numerous individual sequencing reactions. In one embodiment, the array comprises at least 1,000 reaction chambers. In another embodiment, the array comprises greater than 400,000 reaction chambers, preferably between 400,000 and 20,000,000 reaction chambers. In a more preferred embodiment, the array comprises between 1,000,000 and 16,000,000 reaction chambers.

The reaction chambers on the array may take the form of a cavity or well in the substrate material, having a width and depth, into which reactants can be deposited. One or more of the reactants typically are bound to the substrate material in the reaction chamber and the remainder of the reactants are in a medium which facilitates the reaction and which flows through the reaction chamber. When formed as cavities or wells, the chambers are preferably of sufficient dimension and order to allow for (i) the introduction of the necessary reactants into the chambers, (ii) reactions to take place within the chamber and (iii) inhibition of mixing of reactants between chambers. The shape of the well or cavity is preferably circular or cylindrical, but can be multisided so as to approximate a circular or cylindrical shape. In another embodiment, the shape of the well or cavity is substantially hexagonal. The cavity can have a smooth wall surface. In an additional embodiment, the cavity can have at least one irregular wall surface. The cavities can have a planar bottom or a concave bottom. The reaction chambers can be spaced between 5 μm and 200 μm apart. Spacing is determined by measuring the center-to-center distance between two adjacent reaction chambers. Typically, the reaction chambers can be spaced between 10 μm and 150 μm apart, preferably between 50 μm and 100 μm apart. In one embodiment, the reaction chambers have a width in one dimension of between 0.3 μm and 100 μm. The reaction chambers can have a width in one dimension of between 0.3 μm and 20 μm, preferably between 0.3 μm and 10 μm, and most preferably about 6 μm. In another embodiment, the reaction chambers have a width of between 20 μm and 70 μm. Ultimately the width of the chamber may be dependent on whether the nucleic acid samples require amplification. If no amplification is necessary, then smaller, e.g., 0.3 μm is preferred. If amplification is necessary, then larger, e.g., 6 μm is preferred. The depth of the reaction chambers are preferably between 10 μm and 100 μm. Alternatively, the reaction chambers may have a depth that is between 0.25 and 5 times the width in one dimension of the reaction chamber or, in another embodiment, between 0.3 and 1 times the width in one dimension of the reaction chamber.

Any material can be used as the solid support material, as long as the surface allows for stable attachment of the primers and detection of nucleic acid sequences. The solid support material can be planar or can be cavitated, e.g., in a cavitated terminus of a fiber optic or in a microwell etched, molded, or otherwise micromachined into the planar surface, e.g. using techniques commonly used in the construction of microelectromechanical systems. See e.g., Rai-Choudhury, HANDBOOK OF MICROLITHOGRAPHY, MICROMACHINING, AND MICROFABRICATION, VOLUME 1: MICROLITHOGRAPHY, Volume PM39, SPIE Press (1997); Madou, CRC Press (1997), Aoki, Biotech. Histochem. 67: 98-9 (1992); Kane et al., Biomaterials. 20: 2363-76 (1999); Deng et al., Anal. Chem. 72:3176-80 (2000); Zhu et al., Nat. Genet. 26:283-9 (2000). In some embodiments, the solid support is optically transparent, e.g., glass.

In one embodiment, each cavity or reaction chamber of the array contains reagents for analyzing a nucleic acid or protein. Typically those reaction chambers that contain a nucleic acid (not all reaction chambers in the array are required to) contain only a single species of nucleic acid (i.e., a single sequence that is of interest). There may be a single copy of this species of nucleic acid in any particular reaction chamber, or they may be multiple copies. It is generally preferred that a reaction chamber contain at least 100 copies of a nucleic acid sequence, preferably at least 100,000 copies, and most preferably between 100,000 to 1,000,000 copies of the nucleic acid. The ordinarily skilled artisan will appreciate that changes in the number of copies of a nucleic acid species in any one reaction chamber will affect the signal generated in a sequencing reaction utilizing electronic sensing elements as discussed further herein, and thus the number of species can be routinely adjusted to provide more or less signal as is required.

### III. Methods of Sequencing

III.A. Stepwise Electronic Sequencing

In one aspect, the present invention provides methods and compositions for stepwise electronic sequencing in which the sequence of a plurality of template nucleic acids is identified.

In further aspects, methods of the present invention include methods of identifying a sequence of a plurality of template nucleic acids in which a plurality of immobilized clonal populations of primed nucleic acids are provided such that each clonal population is in contact with or proximate to an electronic sensing element. Such clonal populations can be generated using methods known in the art, including without limitation bridge amplification and emulsion amplification methods. See Metzker, Nature Genetics, 2010, Volume 11 for an exemplary discussion of such amplification methods. “Primed nucleic acids” as discussed herein refer to nucleic acids that are in a condition to be replicated and/or extended in a template-directed manner, including without limitation nucleic acids hybridized to a primer that can be extended through the action of a polymerase as well as double stranded nucleic acids comprising a gap or a nick from which sequence-dependent replication can occur. Typically clonal populations are used in stepwise sequencing methods of the invention, but in some cases the stepwise method is performed using a single molecule. The methods of the invention allow for single molecule stepwise sequencing because of the amplification of signal that is obtained by detecting the cleavage of multiple phosphate bonds per incorporation event.

The electronic sensing element for use in methods of the present invention may include without limitation an element that senses ionic changes or pH changes, an element that senses temperature changes, an element that senses changes in magnetic field, a field effect transistor, and an ion sensitive field effect transistor. In exemplary embodiments and as is discussed in further detail below, the electronic sensing element of use in methods of the present invention may include field effect transistors, particularly chemical field effect transistors, which translate a change in ion concentration (including hydrogen ion concentration—also referred to as pH) into an electrical signal.

In further aspects, the methods of the present invention include exposing the plurality of immobilized clonal populations to a first type of nucleoside polyphosphate that comprises a polyphosphate chain of four or more phosphates. The immobilized clonal populations are exposed to the first type of nucleoside polyphosphates under conditions supporting a template directed incorporation of the nucleoside monophosphate portion of the first type of nucleoside polyphosphate. Upon such an incorporation (which, as will be appreciated, occurs if the first type of nucleoside polyphosphate comprises a nucleobase complementary to a base of the template nucleic acid), the alpha-beta phosphate bond of the first type of nucleoside polyphosphate is cleaved by a polymerase enzyme, and one or more other phosphate bonds are cleaved typically by an enzyme such as a phosphatase, although chemical cleavage reactions are also contemplated. The incorporation of the first type of nucleoside polyphosphate thus results in the release of a polyphosphate chain and the cleavage of at least one additional phosphate bond of that polyphosphate chain. Thus, the incorporation of the first type of nucleoside polyphosphate results in the cleavage of at least two phosphate bonds per incorporation event, resulting in the release of at least two protons and the release of at least two phosphate ions per incorporation event. This is an advantage over other electronic sequencing methods known in the art, which utilize standard nucleotides and release only a single hydrogen ion per incorporation event. In further embodiments, a second, third and fourth type of nucleoside polyphosphate is utilized in the above-described methods. The first, second, third, and fourth type of nucleoside polyphosphates will in some embodiments correspond to the nucleobases A, G, T and C, such that repeating the above steps results in identification of the sequence of the template nucleic acids of each of the clonal populations.

As discussed above, the different types of nucleotide analogs of use in the present invention may in some embodiments each have a different number of phosphate groups in the polyphosphate chain, such that each type may be identified from each other type upon incorporation. For example, the different types of nucleotide analogs may each correspond to a nucleobase independently selected from A, G, C, or T (or to one or more modified nucleobases), and each type may be distinguished from the other types based on characteristics such as the signal generated when the nucleotide analog is incorporated during a polymerase reaction. For example, each type of nucleotide analog can in some embodiments have a different number of phosphate groups in the polyphosphate chain, such that, upon incorporation of a particular nucleotide analog type during a polymerization reaction, the signal associated with the resultant cleavage of the phosphate bonds of the polyphosphate chain will identify the incorporated nucleotide analog as having a nucleobase A, C, G, or T. In further embodiments, sequencing reactions discussed herein may utilize 1, 2, 3, 4, 5, 6, 7, 8, 9, 10, 11, 12 or more different types of nucleotide analogs, and in further exemplary embodiments each of the different types of nucleotide analogs has a different number of phosphate groups in their polyphosphate chains.

Although in general the stepwise sequencing methods of the invention utilize one type of nucleoside polyphosphate for each round of incorporation and detection, it will be appreciated that such sequencing methods may also be conducted with multiple (1, 2, 3, 4, 5, 6, 7, 8, 9, 10, 11, 12 or more different types of nucleotide analogs) during each round of incorporation and detection. In further exemplary embodiments, each of the different types nucleotide analogs of use in the sequencing methods discussed herein have a number of phosphate groups independently selected from 3, 4, 5, 6, 7, 8, 9, 10, 11, 12, 13, 14, 15, 16, 17, 18, 19, 20, 21, 22, 23, 24, 25, 26, 27, 28, 29, or 30 phosphate groups.

In further aspects of the invention, each of the clonal populations or isolated single molecules is electrically monitored with the electronic sensing elements to detect whether one or more incorporations of the first type of nucleoside polyphosphate occurs at that clonal population, thereby identifying a nucleotide of the template nucleic acid at that clonal population.

In still further aspects, the exposing and detecting steps are repeated with a second, third and fourth type of nucleoside polyphosphates enough times to identify the sequence of the plurality of template nucleic acids

Detecting the incorporation of the nucleoside polyphosphate in accordance with the methods discussed herein comprises a detection (also referred to herein as sensing) of one or more changes that result from the cleavage of multiple phosphate bonds upon that incorporation. For example, the electronic sensing elements of the invention may sense, without limitation, ionic changes, pH changes, temperature changes, and changes in magnetic field in response to the incorporation of nucleoside polyphosphate.

Electronic sensing elements that detect ionic changes, including changes in hydrogen concentration (i.e., changes in pH) are known in the art. Such electronic sensing elements include without limitation ion-selective electrodes, field effect transistors (FET), ion-sensitive field effect transistors (ISFET), chemical field effect transistors (chemFET), metal-insulator-semiconductor field-effect transistor (MISFET), and metal-oxide-semiconductor field-effect transistors (MOSFET). Such electronic sensing elements can be used to detect changes in ion concentrations that result from incorporation of nucleotide analogs in accordance with the methods described herein and translate that change to an electrical signal (e.g., voltage). Such sensors may also be used to detect changes in temperature that result from incorporation of nucleotide analogs in accordance with the methods described herein.

Electronic sensing elements that detect changes in magnetic strength in response to incorporation of the nucleoside polyphosphate in accordance with the present invention may sense changes in magnetic field that result from magnetic particles that are sensitive to changes in pH or ionic changes in the solution. Thus, when a nucleotide analog is incorporated and two or more phosphate groups are cleaved, the hydrogen ions released from that incorporation event results in a change of pH or change in ionic strength that can cause changes in the magnetic field generated from such magnetic particles. Such particles are known in the art—see for example Banerjee et al., 2008, Nanotechnology, 19(50), which is herein incorporated by reference in its entirety for all purposes and in particular for all teachings related to pH sensitive magnetic particles.

In further embodiments, the identity of the nucleoside polyphosphate incorporated in accordance with the methods discussed herein is determined by the characteristics of the signal detected by the electronic sensing elements. Such characteristics may include without limitation the intensity or other quantification of the amount of the signal as well as the time characteristics of that signal. For example, in embodiments in which it is changes in hydrogen ion concentration that are detected by the electronic sensing element, the amount of hydrogen ion may be detected (e.g., by measuring the pH), or it may be the kinetics of the change in hydrogen ion concentration over time as the polyphosphate chain is cleaved. Since the nucleoside polyphosphates used in the invention contain four or more phosphate groups, multiple phosphate bond cleavages occur with each incorporation event. The measurement of those changes over time (e.g., the kinetics of the cleavage reactions) may in some embodiments be the signal characteristic for identifying the sequence of the template nucleic acids.

In embodiments in which the kinetic change associated with the cleavage of the phosphate bonds is being determined, the kinetics of the phosphate bond cleavage reaction can be adjusted to increase the resolution of detection and allow for detection of individual phosphate cleavage events over time. Methods for controlling the activity of such reactions, including those governed by enzymes such as phosphatases, are known in the art, and generally involve controlling the initiation and the halting of the enzyme reaction, adjusting the concentration of the phosphatase enzyme, adjusting the presence of particular additives that influence the kinetics of the reaction, adjusting the type, concentration, and relative amounts of various cofactors, including metal cofactors, and changing other conditions such as temperature, ionic strength. In further embodiments, the kinetics of the cleavage reaction are adjusted to ensure that phosphate cleavage occurs within enough time to allow the electronic sensing elements to detect the cleavage events before the polyphosphate chain (and the cleaved byproducts) diffuses away from the reaction site.

As will be appreciated, the cleavage of the phosphate bonds in the polyphosphate chain released upon incorporation of the nucleoside monophosphate portion of the nucleoside polyphosphate can be accomplished by any means known in the art. In exemplary embodiments, the cleavage reaction is governed by enzymatic or non-enzymatic processes. For enzymatic processes, any phosphatase (or any other enzyme with phosphatase activity, i.e., the ability to remove a phosphate group from the polyphosphate chain) known in the art can be used. There are a variety of different phosphatases with a wide variety of enzymatic properties that are of use for the sequencing methods described herein, including without limitation any of the phosphoric monoester hydrolases, such as acid phosphatase, alkaline phosphatase, fructose-bisphosphatase, glucose-6-phosphatase, histidinol-phosphatase, 4-nitrophenylphosphatase, nucleotidases, phosphatidate phosphatase, phosphofructokinase-2, phosphoprotein phosphatases, 6-phytase, and Antarctic phosphatase. In exemplary embodiments, alkaline phosphatases, such as shrimp alkaline phosphatase and calf intestinal phosphatase, are of use in accordance with the present invention. In certain specific embodiments, the phosphatase used in methods of the invention is not a pyrophosphatase. For embodiments utilizing non-enzymatic phosphate bond cleavage reactions, a small molecule that binds the terminal phosphate along with a divalent metal (Mg2+ or Mn2+) can be engineered to carry out the hydrolysis reaction.

In embodiments in which an enzyme such as a phosphatase is used in methods of the invention, the enzyme can in exemplary embodiments be disposed close enough to the site at which the nucleoside polyphosphate is incorporated to allow the phosphatase to encounter the released polyphosphate chain and implement the hydrolysis reaction to cleave one or more phosphate bonds of the released polyphosphate. In still further embodiments, the phosphatase may be immobilized at or near the same site at which the clonal population of template nucleic acids is disposed to allow for the cleavage reaction to take place upon incorporation of the nucleoside polyphosphate and release of the polyphosphate chain.

The following discussion provides descriptions of different embodiments of the electronic sensing elements used to conduct the basic steps discussed above. As will be appreciated, each of the following embodiments utilize nucleotide analogs in accordance with the present invention, thus increasing the amount of signal produced with each incorporation event as compared to methods in which nucleoside triphosphates are utilized. As will also be appreciated, although the following embodiments are described primarily in terms of detecting hydrogen ions released by the incorporation events, these embodiments can be readily adjusted by the skilled artisan to detect signals related to changes in any ion concentration, to changes in temperature, and to changes in magnetic field, as discussed above.

In some embodiments, stepwise sequencing methods of the invention are conducted in a semiconductor-based/microfluidic hybrid system that combines microelectronics with a microfluidic system, such as the systems described for example in U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362, each of which is herein incorporated by reference in its entirety for all purposes and in particular for all teachings related to systems, methods and compositions for sequencing pluralities of clonal nucleic acid populations utilizing electronic sensors such as semiconductor-based systems.

Some of the discussion herein for the electronics (including microelectronics) components used in methods of sequencing is in terms of complementary metal-oxide semiconductor (CMOS) technology for purposes of illustration. It should be appreciated, however, that the disclosure is not intended to be limiting in this respect, as other semiconductor-based technologies may be utilized to implement various aspects of the microelectronics portion of the systems discussed herein.

In an exemplary embodiment, the stepwise sequencing methods of the invention utilize nucleoside polyphosphates that comprise a polyphosphate chain of four or more phosphates (or any of the nucleoside polyphosphates discussed in further detail herein) in a system comprising a large sensor array of chemical field-effect transistors (chemFETs), where the individual chemFET sensor elements or “pixels” of the array are configured to detect analyte (e.g., ions, for example hydrogen ions), presence (or absence), analyte levels (or amounts), and/or analyte concentration in an unmanipulated sample, or as a result of chemical and/or biological processes (e.g., chemical reactions, cell cultures, neural activity, nucleic acid sequencing processes, etc.) occurring in proximity to the array. Examples of chemFETs encompassed by methods of the present invention include, but are not limited to, ISFETs and EnFETs. In one exemplary implementation, one or more microfluidic structures is/are fabricated above the chemFET sensor array to provide for containment and/or confinement of a biological or chemical reaction in which an analyte of interest may be produced or consumed, as the case may be. For example, in one embodiment, the microfluidic structure(s) may be configured as one or more “wells” (e.g., small reaction chambers or “reaction wells”) disposed above one or more sensors of the array, such that the one or more sensors over which a given well is disposed detect and measure analyte presence, level, and/or concentration in the given well.

In exemplary embodiments, the invention encompasses a system for high-throughput sequencing comprising at least one two-dimensional array of reaction chambers, where each reaction chamber is coupled to a chemFET and each reaction chamber is no greater than 10 μm³ (i.e., 1 μL) in volume. Preferably, each reaction chamber is no greater than 0.34 pL, and more preferably no greater than 0.096 pL or even 0.012 pL in volume. A reaction chamber can optionally be 2², 3², 4², 5², 6², 7², 8², 9², or 10² square microns in cross-sectional area at the top. Preferably, the array has at least 100, 1,000, 10,000, 100,000, or 1,000,000 reaction chambers. The reaction chambers may be capacitively coupled to the chemFETs, and preferably are capacitively coupled to the chemFETs.

In still further embodiments, the stepwise sequencing methods of the present invention may be conducted in a device comprising an array of chemFETs with an array of microfluidic reaction chambers and/or a semiconductor material coupled to a dielectric material. Such devices are discussed for example in U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362, each of which is herein incorporated by reference in its entirety for all purposes and in particular for all teachings related to sequencing and/or detection of byproducts of biological reactions using such devices and associated electronic sensing elements.

In yet further embodiments, the methods of the invention conducted in any of the above-described systems or on platforms known in the art may be automated via robotics. In addition, the information obtained via the signal from the chemFET may be provided to a personal computer, a personal digital assistant, a cellular phone, a video game system, or a television so that a user can monitor the progress of reactions remotely.

As discussed above, in some embodiments an analyte of particular interest is hydrogen ions, and methods of sequencing as discussed herein can utilize large scale ISFET arrays specifically configured to measure ionic concentration or pH. In other embodiments, the chemical reactions being monitored may relate to DNA synthesis processes, or other chemical and/or biological processes, and chemFET arrays may be specifically configured to measure pH or the concentration of one or more other analytes that provide relevant information relating to a particular chemical process of interest. In various aspects, the chemFET arrays are fabricated using conventional CMOS processing technologies, and are particularly configured to facilitate the rapid acquisition of data from the entire array (scanning all of the pixels to obtain corresponding pixel output signals). Such arrays are known in the art and described for example in US 2009/0026082, which is hereby incorporated by reference in its entirety for all purposes and in particular for all teachings related to methods and devices for analyte measurements, particularly for analyte measurements related to DNA polymerase and/or sequencing reactions.

With respect to analyte detection and measurement, it should be appreciated that in various embodiments discussed herein, one or more analytes measured by a chemFET array according to the present disclosure may include any of a variety of chemical substances that provide relevant information regarding a chemical process or chemical processes of interest (e.g., binding of multiple nucleic acid strands, binding of an antibody to an antigen, etc.). In preferred embodiments, the analyte detected is associated with incorporation of a nucleotide analog as discussed above. Such an analyte may include a change in hydrogen ion concentration resulting from incorporation of the nucleotide analog or may include another analyte (such as another ion or temperature) affected by the incorporation of the nucleoside polyphosphate and subsequent cleavage of multiple phosphate bonds. In some aspects, the ability to measure levels or concentrations of one or more analytes, in addition to merely detecting the presence of an analyte, provides valuable information in connection with the chemical process or processes. In other aspects, mere detection of the presence of an analyte or analytes of interest may provide valuable information. In further embodiments and as discussed herein, the identity of the analyte can be determined by the characteristics of the signal detected by the electronic sensing elements. Such characteristics may include without limitation the intensity or other quantification of the amount of the signal or the kinetics of that signal.

Devices for stepwise sequencing in accordance with any of the methods described herein, including chemFET arrays described herein and known in the art (see for example in U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362, each of which is herein incorporated by reference in its entirety for all purposes) according to various inventive embodiments of the present invention may be configured for sensitivity to any one or more of a variety of analytes/chemical substances. In one embodiment, one or more chemFETs of an array may be particularly configured for sensitivity to one or more analytes representing one or more binding events (e.g., associated with a nucleic acid sequencing process), and in other embodiments different chemFETs of a given array may be configured for sensitivity to different analytes. For example, in one embodiment, one or more sensors (pixels) of the array may include a first type of chemFET configured to be chemically sensitive to a first analyte, and one or more other sensors of the array may include a second type of chemFET configured to be chemically sensitive to a second analyte different from the first analyte. In one exemplary implementation, the first analyte may represent a first binding event associated with a nucleic acid sequencing process, and the second analyte may represent a second binding event associated with the nucleic acid sequencing process. Of course, it should be appreciated that more than two different types of chemFETs may be employed in any given array to detect and/or measure different types of analytes/binding events. In general, it should be appreciated in any of the embodiments of sensor arrays discussed herein that a given sensor array may be “homogeneous” and include chemFETs of substantially similar or identical types to detect and/or measure a same type of analyte (e.g., pH or other ion concentration), or a sensor array may be “heterogeneous” and include chemFETs of different types to detect and/or measure different analytes.

In a further aspect, the methods of the present invention include methods of sequencing a nucleic acid where the methods include the step of disposing a plurality of template nucleic acids into a plurality of reaction chambers, wherein the plurality of reaction chambers is in contact with or proximate to a chemical-sensitive field effect transistor (chemFET) array comprising at least one chemFET for each reaction chamber, and wherein each of the template nucleic acids is hybridized to a sequencing primer and is bound to a polymerase. Such methods further include a step of synthesizing a new nucleic acid strand by incorporating one or more known nucleoside polyphosphates containing a phosphate chain of 4 or greater (or any of the nucleoside polyphosphates discussed herein) sequentially at the 3′ end of the sequencing primer and detecting the incorporation of the one or more known nucleoside polyphosphates by the generation of sequencing reaction byproduct. In some embodiments, the chemFET array comprises more than 256 sensors and/or a center-to-center distance between adjacent reaction chambers (or “pitch”) of 1-10 μm.

In a further aspect and in accordance with any of the above, the invention includes methods for sequencing a nucleic acid in which a target nucleic acid is fragmented to generate a plurality of fragmented nucleic acids. In this aspect, each of the plurality of fragmented nucleic acids can be attached to individual beads to generate a plurality of beads each attached to a single fragmented nucleic acid. The number of fragmented nucleic acids on each bead is then increased by amplifying the number of fragmented nucleic acids on each bead. The plurality of beads attached to amplified fragmented nucleic acids is then delivered to a chemical-sensitive field effect transistor (chemFET) array having a separate reaction chamber for each sensor in the array, wherein only one bead is situated in each reaction chamber. Sequencing reactions can then be performed simultaneously in the plurality of reaction chambers in accordance with any of the methods described herein.

In a further embodiment and in accordance with any of the above, the invention includes methods for sequencing a nucleic acid in which a target nucleic acid is fragmented to generate a plurality of fragmented nucleic acids. Each of these fragmented nucleic acids is amplified separately in the presence of a bead and the amplified copies of the fragmented nucleic acid are attached to the bead, thereby producing a plurality of beads each having attached multiple identical copies of a fragmented nucleic acid. The plurality of beads each having attached multiple identical copies of a fragmented nucleic acid are delivered to a chemical-sensitive field effect transistor (chemFET) array having a separate reaction chamber for each chemFET sensor in the array, wherein only one bead is situated in each reaction chamber. Sequencing reactions can then be performed simultaneously in the plurality of reaction chambers.

As discussed above, in some embodiments, the invention provides a method for sequencing a nucleic acid comprising disposing a plurality of template nucleic acids into a plurality of reaction chambers, where the plurality of reaction chambers is in contact with or proximate to an chemical-sensitive field effect transistor (chemFET) array comprising at least one chemFET for each reaction chamber, and where each of the template nucleic acids is hybridized to a sequencing primer and is bound to a polymerase. The method further includes a step of synthesizing a new nucleic acid strand by incorporating one or more known nucleotide analogs sequentially at the 3′ end of the sequencing primer, and detecting a change in the level of a sequencing byproduct as an indicator of incorporation of the one or more known nucleotide analogs. The plurality of template nucleic acids may in some embodiments be clonal populations of amplified template fragments, where each clonal population is in a separate reaction chamber. In further embodiments, the clonal population of template nucleic acids is attached to a bead.

The change in the level of the sequencing byproduct detected in any of the aspects and embodiments described above may in further embodiments be an increase or a decrease in a level relative to that level prior to incorporation of the one or more known nucleoside polyphosphates. The change in the level may be read as a change in current at a chemFET sensor or a change in pH, but it is not so limited. In exemplary embodiments, the sequencing byproduct is inorganic pyrophosphate (PPi). In a related embodiment, PPi is detected by binding to a PPi receptor on the surface of one or more chemFET sensors in the array.

In still further embodiments, the sequencing reaction byproduct is inorganic pyrophosphate (PPi). In some embodiments, PPi is measured directly. In some embodiments, the PPi is measured in the absence of a PPi receptor. In some embodiments, the sequencing reaction byproduct is hydrogen ions. In some embodiments, the sequencing reaction byproduct is inorganic phosphate (Pi). In still other embodiments, the chemFET detects changes in any combination of the byproducts, optionally in combination with other parameters, as described herein.

In some aspects, the invention provides a method for sequencing a nucleic acid comprising disposing a plurality of template nucleic acids into a plurality of reaction chambers, wherein the plurality of reaction chambers is in contact with or proximate to an chemical-sensitive field effect transistor (chemFET) array comprising at least one chemFET for each reaction chamber, and wherein each of the template nucleic acids is hybridized to a sequencing primer and is bound to a polymerase, synthesizing a new nucleic acid strand by incorporating one or more types of nucleotide analogs sequentially at the 3′ end of the sequencing primer, directly detecting release of inorganic pyrophosphate (PPi) as an indicator of incorporation of the one or more types of nucleotide analogs. In some embodiments, the PPi is directly detected by binding to a PPi receptor immobilized on the chemFET. In some embodiments, the PPi is directly detected by the chemFET in the absence of a PPi receptor.

Various embodiments apply equally to the methods disclosed herein and they are recited once for brevity. In some embodiments, the center-to-center distance between adjacent reaction chambers is about 2-9 μm, about 2 μm, about 5 μm, or about 9 μm. In some embodiments, the chemFET array comprises more than 256 sensors (and optionally more than 256 corresponding reaction chambers (or wells), more than 10³ sensors (and optionally more than 10³ corresponding reaction chambers), more than 10⁴ sensors (and optionally more than 10⁴ corresponding reaction chambers), more than 10⁵ sensors (and optionally more than 10⁵ corresponding reaction chambers), or more than 10⁶ sensors (and optionally more than 10⁶ corresponding reaction chambers). In some embodiments, the chemFET array comprises at least 512 rows and at least 512 columns of sensors.

In further embodiments, the electronic sensing elements include any sensor architecture known in the art, including those for example described in U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362, each of which is herein incorporated by reference in its entirety for all purposes and in particular for all teachings related to electronic sensors and sensing elements for detection of the byproducts of biological reactions, including sequencing reactions.

In some embodiments, the electronic sensing elements of use in the methods of the present invention include a scalable ISFET sensor architecture using electronic addressing common in modern CMOS imagers. Such integrated circuits may in some embodiments include an array of sensor elements, each with a single floating gate connected to an underlying ISFET. In further embodiments, confinement of the reactants of the biological reactions under study (including DNA sequencing) is accomplished using a well formed by adding a dielectric layer over the electronics and etching to the sensor plate. In specific embodiments, a 3.5-μm-diameter well formed by adding a 3-μm-thick dielectric layer over the electronics and etching to the sensor plate. A tantalum oxide layer can then provide for proton sensitivity. Specifics of such architectures can be in accordance with embodiments known in the art and described for example in U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362, each of which is herein incorporated by reference in its entirety for all purposes and in particular for all teachings related to electronic sensors and sensing elements for detection of the byproducts of biological reactions, including sequencing reactions.

In further exemplary embodiments, the electronic sensors of use in methods of the invention comprise semiconductor electronics integrated with a sensor array, such as those described for example in any of U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362. The sensor and underlying electronics provide a direct transduction from the incorporation event to an electronic signal. Unlike light-based sequencing technology, we do not use the elements of the array to collect photons and form a larger image to detect the incorporation of a base, each sensor independently and directly monitors the hydrogen ions released during nucleotide incorporation. Ion chips can be manufactured on wafers, cut into individual die and packaged with a disposable polycarbonate flow cell that isolates the fluids to regions above the sensor array and away from the supporting electronics to provide convenient sample loading as well as electrical and fluidic interfaces to the sequencing instrument. Increasing the numbers of sensors per chip can be achieved by increasing the die area, and then by increasing the density of the sensors by reducing the number of transistors per sensor. In an exemplary embodiment, 1.3 μm wells are aligned to sensors enabling generation of high-quality sequence reads.

In further aspects, the present invention provides integrated systems for conducting the stepwise sequencing methods described herein. Such systems in some embodiments comprise components for detecting both optical and electronic signals. In further embodiments, the systems comprise no optical components and include primarily an electronic reader board that interfaces with the chip, a microprocessor for signal processing, and a fluidics system to control the flow of reagents over the chip.

In further exemplary embodiments, the methods of the present invention include preparing genomic DNA by methods known in the art, including fragmenting the DNA and clonally amplifying the DNA onto a substrate such as a bead. In certain embodiments, the fragments are first ligated to one or more adaptors, and the adaptor-ligated fragments are then clonally amplified. In embodiments in which beads are used, template-bearing beads can be enriched through methods such as a magnetic bead-based process. Sequencing primers and DNA polymerase are then bound to the templates and pipetted into the chip's loading port. Individual beads are loaded into individual sensor wells. In further embodiments, well depth is selected to allow only a single bead to occupy a well.

In further embodiments, different types of nucleotide analogs are provided in a stepwise fashion. When the nucleotide analog in the flow is complementary to the template base directly downstream of the sequencing primer, the nucleotide is incorporated into the nascent strand by the bound polymerase. This increases the length of the sequencing primer by one base and results in the hydrolysis of the incoming nucleotide analog, which causes the net liberation of multiple protons for each nucleotide analog incorporated during that flow, because, as is described herein, the nucleotide analog comprises multiple phosphate groups in the polyphosphate chain. The release of the proton produces a shift in the pH of the surrounding solution proportional to the number of nucleotide analogs incorporated in the flow (0.02 pH units per single base incorporation). This can be detected by the sensor on the bottom of each well, converted to a voltage and digitized by off-chip electronics. After the flow of each nucleotide, a wash can in further embodiments be used to ensure nucleotides do not remain in the well. The small size of the wells allows diffusion into and out of the well on the order of a one-tenth of a second and eliminates the need for enzymatic removal of reagents

In further exemplary embodiments, to change raw voltages from the electronic sensors into base calls, signal-processing software can be used to convert the raw data into measurements of incorporation in each well for each successive nucleotide flow using a physical model. Sampling the signal at high frequency relative to the time of the incorporation signal allows signal averaging to improve the signal to noise ratio (SNR). The use of the nucleotide analogs of the present invention with the 4 or more phosphate groups further increases the SNR and may obviate or lessen the need for signal averaging. Further signal processing techniques are known in the art and described for example in U.S. Pat. No. 7,335,762; U.S. Pat. No. 8,349,167; US2013/0017959; US2013/0012399; WO2011/120964; US2009/0026082, US2009/0127589, US2010/0301398, US2010/0300895, US2010/0300559, US2010/0197507, US 2010/0137143; WO2012/045889; EP2304420; Rothberg et al., 2011, Nature, 475:348-352; Credo et al., 2012, Analyst, 137(6): 1351-1362.

In a further aspect, the present invention provides a method of identifying a sequence of a plurality of template nucleic acids, in which a plurality of immobilized clonal populations of primed nucleic acid templates is provided, where each clonal population is proximate to an electronic sensing element. In such a method, the plurality of immobilized clonal populations is exposed to a first type of nucleoside polyphosphate under conditions supporting a template directed incorporation of a nucleoside monophosphate portion of the first type of nucleoside polyphosphate. The first type of nucleoside polyphosphate will in this aspect include a polyphosphate chain of three or more phosphates and a terminal blocking group, and the incorporation reaction is carried out in the presence of a phosphatase enzyme. Such a phosphatase enzyme may include without limitation a shrimp alkaline phosphatase. The terminal blocking group on the polyphosphate chain prevents phosphatase cleavage of the nucleoside polyphosphate until the incorporation event, and then upon incorporation of the nucleoside polyphosphate, the cleavage of an alpha-beta phosphate bond and at least one additional phosphate bond of the incorporated nucleoside polyphosphate occurs. The terminal blocking group may in some embodiments comprise without limitation a member selected from a methyl group, an amino hexyl group, a dye, an adduct, and a linker. The method further includes electrically monitoring each of the clonal populations with the electronic sensing elements to detect whether one or more incorporations of the first type of nucleoside polyphosphate occurs at that clonal population. The incorporation reaction and electrical monitoring steps are then repeated with second, third and fourth types of nucleoside polyphosphates for a number of times to thereby identify the sequence of the plurality of template nucleic acids. In further embodiments, the number of immobilized clonal populations of primed nucleic acid templates is between 1,000 and 10 million.

In accordance with the above aspect, the electronic sensing elements used in the method to electrically monitor each of the clonal populations will in certain embodiments sense the ionic changes that result from the cleavage of the phosphate bonds. Such an electronic sensing element could in a non-limiting embodiment include an ion sensitive field effect transistor (ISFET). In still further embodiments, the clonal populations of the primed nucleic acid templates are provided on beads. In yet further embodiments, the polyphosphate chain has 4, 5, 6, 7, 8, 9, 10, 11, or 12 phosphates. In still further embodiments, the first, second, third, and fourth types of nucleoside polyphosphates each correspond to a nucleobase independently selected from A, G, C, or T.

III. B. Single-Molecule Electronic Sequencing

In some aspects, the present invention provides methods for single-molecule electronic sequencing. Such methods include providing a plurality of individually resolvable single-molecule polymerase-template complexes, where each complex includes a template nucleic acid, a polymerase enzyme and a primer. Each complex is also associated with an electronic sensing element. In some cases, the single molecule method can be carried out in a stepwise fashion as described above. In other cases, the single molecule sequencing reaction can be carried out in real time. As with the stepwise sequencing methods discussed above, the electronic sensing element may include without limitation an element that senses ionic changes or pH changes, an element that senses temperature changes, an element that senses changes in magnetic field, a field effect transistor, and an ion sensitive field effect transistor.

In further aspects, the single molecule real time sequencing methods of the invention include a step of exposing the complexes to two or more types of nucleoside polyphosphates, where the two or more types of nucleoside polyphosphates each comprises a phosphate chain of four or more phosphates. In addition, each type of nucleoside polyphosphate has a different number of phosphates.

As discussed above, the different types of nucleotide analogs of use in the present invention may in some embodiments each have a different number of phosphate groups in the polyphosphate chain, such that each type may be identified from each other type upon incorporation. For example, the different types of nucleotide analogs may each correspond to a nucleobase independently selected from A, G, C, or T (or to one or more modified nucleobases), and each type may be distinguished from the other types based on characteristics such as the signal generated when the nucleotide analog is incorporated during a polymerase reaction. Each type of nucleotide analog can in some embodiments have a different number of phosphate groups in the polyphosphate chain, such that, upon incorporation of a particular nucleotide analog type during a polymerization reaction, the signal associated with the resultant cleavage of the phosphate bonds of the polyphosphate chain will identify the incorporated nucleotide analog as having a nucleobase A, C, G, or T. In further embodiments, sequencing reactions discussed herein may utilize 1, 2, 3, 4, 5, 6, 7, 8, 9, 10, 11, 12 or more different types of nucleotide analogs, and in further exemplary embodiments each of the different types of nucleotide analogs has a different number of phosphate groups in their polyphosphate chains. In further exemplary embodiments, each of the different types nucleotide analogs of use in the sequencing methods discussed herein have a number of phosphate groups independently selected from 4, 5, 6, 7, 8, 9, 10, 11, 12, 13, 14, 15, 16, 17, 18, 19, 20, 21, 22, 23, 24, 25, 26, 27, 28, 29, 30 phosphate groups.

In further aspects, the step of exposing the complexes to two or more types of nucleoside polyphosphates is carried out under conditions supporting template dependent primer extension through multiple incorporation reactions. Each incorporation reaction results in the cleavage of an alpha-beta phosphate bond and at least one additional phosphate bond of the polyphosphate chain of the incorporated nucleoside polyphosphates. Thus, as with the stepwise sequencing methods discussed above, the real-time single molecule sequencing methods of the present invention result in the cleavage of multiple phosphate bonds per incorporation event—as a result, any signal associated with the cleavage of the multiple phosphate bonds is larger than would be possible for incorporation events in which only a single phosphate bond is cleaved. As will be appreciated, the exposing step may be carried out with 2, 3, 4, 5, 6, 7, 8, 9, 10, 11, 12 or more different types of nucleotide analogs. In further exemplary embodiments, each of the different types nucleotide analogs of use in the sequencing methods discussed herein have a number of phosphate groups independently selected from 4, 5, 6, 7, 8, 9, 10, 11, 12, 13, 14, 15, 16, 17, 18, 19, 20, 21, 22, 23, 24, 25, 26, 27, 28, 29, 30 phosphate groups.

The cleavage of the phosphate bonds is generally accomplished by an enzyme such as a phosphatase, although, as is discussed above and in further detail herein, chemical cleavage reactions are also contemplated. As will be appreciated, the cleavage of the phosphate bonds in the polyphosphate chain released upon incorporation of the nucleoside monophosphate portion of the nucleoside polyphosphate can be accomplished by any means known in the art. For enzymatic processes, any phosphatase (or any other enzyme with phosphatase activity, i.e., the ability to remove a phosphate group from the polyphosphate chain) known in the art can be used. There are a variety of different phosphatases with a wide variety of enzymatic properties that are of use for the sequencing methods described herein, including without limitation any of the phosphoric monoester hydrolases, such as acid phosphatase, alkaline phosphatase, fructose-bisphosphatase, glucose-6-phosphatase, histidinol-phosphatase, 4-nitrophenylphosphatase, nucleotidases, phosphatidate phosphatase, phosphofructokinase-2, phosphoprotein phosphatases, 6-phytase, and Antarctic phosphatase. In exemplary embodiments, alkaline phosphatases, such as shrimp alkaline phosphatase and calf intestinal phosphatase, are of use in accordance with the present invention. In certain specific embodiments, the phosphatase used in methods of the invention is not a pyrophosphatase. For embodiments utilizing non-enzymatic phosphate bond cleavage reactions, a small molecule that binds the terminal phosphate along with a divalent metal (Mg2+ or Mn2+) can be engineered to carry out the hydrolysis reaction.

The phosphate bond cleavages are detected by the electronic sensing elements identify the types of nucleoside polyphosphates incorporated in the incorporation reactions, and thereby sequence the plurality of template nucleic acids. This detecting step includes using one or more characteristics of the signals generated by the phosphate bond cleavages to identify the type of nucleoside polyphosphates incorporated in the incorporation reactions.

As with the stepwise sequencing methods discussed above, detecting the incorporation of the nucleoside polyphosphate in accordance with the single-molecule sequencing methods discussed herein comprises a detection (also referred to herein as sensing) of one or more changes that result from the cleavage of multiple phosphate bonds upon that incorporation. For example, the electronic sensing elements of the invention may sense without limitation ionic changes, pH changes, temperature changes, and changes in magnetic field in response to the incorporation of nucleoside polyphosphate.

Electronic sensing elements that detect ionic changes, including changes in hydrogen concentration (i.e., changes in pH) are known in the art. Such electronic sensing elements include without limitation ion-selective electrodes, field effect transistors (FET), ion-sensitive field effect transistors (ISFET), chemical field effect transistors (chemFET), metal-insulator-semiconductor field-effect transistor (MISFET), and metal-oxide-semiconductor field-effect transistors (MOSFET). Such electronic sensing elements can be used to detect changes in ion concentrations that result from incorporation of nucleotide analogs in accordance with the methods described herein and translate that change to an electrical signal (e.g., voltage). Such sensors may also be used to detect changes in temperature that result from incorporation of nucleotide analogs in accordance with the methods described herein.

Electronic sensing elements that detect changes in magnetic strength in response to incorporation of the nucleoside polyphosphate in accordance with the present invention may sense changes in magnetic field that result from magnetic particles that are sensitive to changes in pH. Thus, when a nucleotide analog is incorporated and two or more phosphate groups are cleaved, the hydrogen ions released from that incorporation event results in a change of pH that can cause changes in the magnetic field generated from such magnetic particles. Such particles are known in the art—see for example Banerjee et al., 2008, Nanotechnology, 19(50), which is herein incorporated by reference in its entirety for all purposes and in particular for all teachings related to pH sensitive magnetic particles.

In further embodiments, the identity of the nucleoside polyphosphate incorporated in accordance with the methods discussed herein is determined by the characteristics of the signal detected by the electronic sensing elements. Such characteristics may include without limitation the intensity or other quantification of the amount of the signal or the kinetics of that signal. For example, in embodiments in which it is changes in hydrogen ion concentration that are detected by the electronic sensing element, the amount of hydrogen ion may be detected (e.g., by measuring the pH), or it may be the kinetics of the change in hydrogen ion as the polyphosphate chain is cleaved. Since the nucleoside polyphosphates used in the invention contain four or more phosphate groups, multiple phosphate bond cleavages occur with each incorporation event. The measurement of those changes over time (e.g., the kinetics of the cleavage reactions) may in some embodiments be the characteristic used to identify the sequence of the template nucleic acids.

In embodiments in which it is the kinetic change associated with the cleavage of the phosphate bonds that is being determined, the kinetics of the phosphate bond cleavage reaction can be adjusted to increase the resolution of detection and allow for detection of individual phosphate cleavage events over time. Methods for controlling the activity of such reactions, including those governed by enzymes such as phosphatases, are known in the art, and generally involve controlling the initiation and the halting of the enzyme reaction, adjusting the concentration of the phosphatase enzyme, to adjust the speed at which the cleavage reaction occurs, including without limitation adjusting the presence of particular additives that influence the kinetics of the reaction, and the type, concentration, and relative amounts of various cofactors, including metal cofactors and changing other conditions such as temperature, ionic strength. In further embodiments, the kinetics of the cleavage reaction are adjusted to ensure that the cleavage occurs within enough time to allow the electronic sensing elements to detect the cleavage events before the polyphosphate chain diffuses away from the reaction site.

In still further embodiments, an engineered phosphate binding protein that has been fluorescently labeled with MDCC (7-Diethylamino-3-((((2-Maleimidyl)ethyl)amino)carbonyl)coumarin) is utilized for identifying the type of nucleoside polyphosphate incorporated by the sequencing reactions discussed above. Upon binding to phosphate, the protein undergoes a large conformational change and the resulting quantum yield increases by greater than >10×. This protein can thus be used to provide an optical readout for the amount of phosphate liberated upon incorporation and hydrolysis. Thus, as discussed herein, different types of nucleotide analogs that have different numbers of phosphate groups can be identified based on the signal characteristic of the intensity or the kinetics of the conformational change of the MDCC-labeled phosphate binding protein in response to the cleavage of the polyphosphate chain in accordance with the methods described herein. Such a sensor is known in the art and described for example in Brune M, et al. (1994) Direct, real-time measurement of rapid inorganic phosphate release using a novel fluorescent probe and its application to actomyosin subfragment 1 ATPase. Biochemistry 33:8262-8271, which is herein incorporated by reference in its entirety for all purposes and in particular for all teachings related to measurement of phosphate release.

As will be appreciated, the cleavage of the phosphate bonds upon incorporation of the nucleoside polyphosphate can be accomplished by any means known in the art. In exemplary embodiments, the cleavage reaction is governed by enzymatic or non-enzymatic processes. For enzymatic processes, any phosphatase known in the art can be used. There are a variety of different phosphatases with a wide variety of enzymatic properties that are of use for the sequencing methods described herein. In exemplary embodiments, alkaline phosphatases, such as shrimp alkaline phosphatase, are use in accordance with the present invention. For embodiments utilizing non-enzymatic phosphate bond cleavage reactions, a small molecule that binds the terminal phosphate along with a divalent metal (Mg2+ or Mn2+) can be engineered to carry out the hydrolysis reaction.

In embodiments in which an enzyme such as a phosphatase is used in methods of the invention, the enzyme can in exemplary embodiments be disposed close enough to the site at which the nucleoside polyphosphate is incorporated to allow the phosphatase to encounter the released polyphosphate chain and implement the hydrolysis reaction to cleave one or more phosphate bonds of the released polyphosphate. In still further embodiments, the phosphatase may be immobilized at or near the same site at which the single-molecule polymerase-template complex is disposed to allow for the cleavage reaction to take place upon incorporation of the nucleoside polyphosphate and release of the polyphosphate chain.

Any of the arrays and substrates discussed above for the stepwise sequencing methods is also suitable for use with single-molecule electronic sequencing methods.

In further embodiments, the methods of the present invention include steps from any single molecule sequencing methods known in the art, wherein those methods utilize the nucleoside polyphosphates having four or more phosphate groups, such that each incorporation event results in a larger signal than would be possible with the use of standard nucleoside triphosphates. Single molecule sequencing applications are well known and well characterized in the art. See, e.g., Rigler, et al., DNA-Sequencing at the Single Molecule Level, Journal of Biotechnology, 86(3): 161 (2001); Goodwin, P. M., et al., Application of Single Molecule Detection to DNA Sequencing. Nucleosides & Nucleotides, 16(5-6): 543-550 (1997); Howorka, S., et al., Sequence-Specific Detection of Individual DNA Strands using Engineered Nanopores, Nature Biotechnology, 19(7): 636-639 (2001); Meller, A., et al., Rapid Nanopore Discrimination Between Single Polynucleotide Molecules, Proceedings of the National Academy of Sciences of the United States of America, 97(3): 1079-1084 (2000); Driscoll, R. J., et al., Atomic-Scale Imaging of DNA Using Scanning Tunneling Microscopy. Nature, 346(6281): 294-296 (1990).

In further embodiments, methods of single molecule sequencing known in the art include detecting individual nucleotides as they are incorporated into a primed template, i.e., sequencing by synthesis. Such methods often utilize exonucleases to sequentially release individual fluorescently labeled bases as a second step after DNA polymerase has formed a complete complementary strand. See Goodwin et al., “Application of Single Molecule Detection to DNA Sequencing,” Nucleos. Nucleot. 16: 543-550 (1997).

In some cases, individual complexes may be provided within separate discrete regions of a support. For example, in some cases, individual complexes may be provided within individual confinement structures, such as zero-mode waveguide cores or any of the reaction chambers discussed above in the stepwise sequencing section. Examples of waveguides and processes for immobilizing individual complexes therein are described in, e.g., Published International Patent Application No. WO 2007/123763, the full disclosure of which is incorporated herein by reference in its entirety for all purposes and in particular for all teachings related to immobilizing complexes.

In preferred aspects, the single-molecule polymerase-template complexes are provided immobilized upon solid supports, and preferably, upon supporting substrates. The complexes may be coupled to the solid supports through one or more of the different groups that make up the complex. For example, in the case of nucleic acid polymerization complexes, attachment to the solid support may be through an attachment with one or more of the polymerase enzyme, the primer sequence and/or the template sequence in the complex. Further, the attachment may comprise a covalent attachment to the solid support or it may comprise a non-covalent association. For example, in particularly preferred aspects, affinity based associations between the support and the complex are envisioned. Such affinity associations include, for example, avidin/streptavidin/neutravidin associations with biotin or biotinylated groups, antibody/antigen associations, GST/glutathione interactions, nucleic acid hybridization interactions, and the like. In particularly preferred aspects, the complex is attached to the solid support through the provision of an avidin group, e.g., streptavidin, on the support, which specifically interacts with a biotin group that is coupled to the polymerase enzyme.

Methods of providing binding groups on the substrate surface that result in the immobilization of complexes are described in, e.g., published U.S. Patent Application No. 2007-0077564, and WO 2007123763, each of which is incorporated herein by reference in its entirety for all purposes and in particular for all teachings related to immobilizing single-molecule polymerase-template complexes.

In some aspects, the present invention includes methods of analyzing the sequence of template nucleic acids isolated in accordance with the methods described herein. In such aspects, the sequence analysis employs template dependent synthesis in identifying the nucleotide sequence of the template nucleic acid. Nucleic acid sequence analysis that employs template dependent synthesis identifies individual bases, or groups of bases, as they are added during a template mediated synthesis reaction, such as a primer extension reaction, where the identity of the base is required to be complementary to the template sequence to which the primer sequence is hybridized during synthesis. Other such processes include ligation driven processes, where oligo- or polynucleotides are complexed with an underlying template sequence, in order to identify the sequence of nucleotides in that sequence. Typically, such processes are enzymatically mediated using nucleic acid polymerases, such as DNA polymerases, RNA polymerases, reverse transcriptases, and the like, or other enzymes such as in the case of ligation driven processes, e.g., ligases.

Sequence analysis using template dependent synthesis can include a number of different processes. For example, in embodiments utilizing sequence by synthesis processes, individual nucleotide analogs are identified iteratively as they are added to the growing primer extension product.

In further embodiments, a sequence by synthesis process that identifies the incorporation of a nucleotide analog by assaying the resulting synthesis mixture for the presence of by-products of the sequencing reaction, namely a released polyphosphate chain comprising three or more phosphate groups. In particular, a primer/template/polymerase complex is contacted with a single type of nucleotide analog. If that nucleotide analog is incorporated, the polymerization reaction cleaves the nucleotide analog between the α and β phosphates of the polyphosphate chain, releasing the remaining chain of phosphate groups. The presence of the released phosphate chain is then identified using the electronic sensing methods described above. Following appropriate washing steps, the various types of nucleotide analogs can be cyclically contacted with the complex to sequentially identify subsequent bases in the template sequence. This sequencing method is analogous to pyrophosphate sequencing methods known in the art (See, e.g., U.S. Pat. No. 6,210,891, incorporated herein by reference in its entirety for all purposes, and in particular for all teachings related to nucleic acid sequencing).

In yet a further embodiment, the incorporation of the different types of nucleotide analogs is observed in real time as template dependent synthesis is carried out. In particular, an individual immobilized primer/template/polymerase complex is observed as the nucleotide analogs are incorporated and two or more phosphate bonds are cleaved, permitting real time identification of each added analog as it is added. Observation of individual molecules in accordance with the present invention typically involves the use of electronic sequencing methods described herein, including any of the arrays of chemFET and ISFET sensors discussed above for stepwise sequencing. In specific embodiments, confining the complex in a reaction chamber allows the creation of a monitored region in which randomly diffusing polyphosphate chains are present for a short period of time, during which the phosphate bonds of those polyphosphate chains are cleaved. This results in a characteristic signal associated with the incorporation event, which is also characterized by a signal profile that is characteristic of the base being added.

For a number of approaches, e.g., single molecule methods as described above, it is generally desirable to provide the nucleic acid synthesis complexes in individually resolvable configurations, such that the synthesis reactions of a single complex can be monitored. As discussed above, providing such complexes in individually resolvable configuration can be accomplished through a number of mechanisms. Further exemplary embodiments include providing a dilute solution of complexes on a substrate surface suited for immobilization, one will be able to provide individually resolvable complexes (See, e.g., European Patent No. 1105529 to Balasubramanian, et al., which is incorporated herein by reference in its entirety for all purposes, and in particular for all teachings related to single molecule sequencing methods.) Alternatively, one may provide a low density activated surface to which complexes are coupled (See, e.g., Published International Patent Application No. WO 2007/041394, the full disclosure of which is incorporated herein by reference in its entirety for all purposes). Such individual complexes may be provided on planar substrates or otherwise incorporated into other structures, e.g., zero mode waveguides or waveguide arrays, to facilitate their observation.

In accordance with any of the above, in one aspect, the present invention provides a method of identifying a sequence of a plurality of template nucleic acids that includes the step of providing a plurality of single-molecule polymerase-template complexes, where each complex includes a template nucleic acid, a polymerase enzyme and a primer and each complex is associated with an electronic sensing element. In this aspect, the complexes are exposed to two or more types of nucleoside polyphosphates, and the two or more types of nucleoside polyphosphates each comprises a phosphate chain of three or more phosphates and a terminal blocking group. In addition, each type of nucleoside polyphosphate has a different number of phosphates. Exposing the complexes to the nucleoside polyphosphates is conducted under conditions supporting template dependent primer extension through multiple incorporation reactions. In addition, in this aspect, the incorporation reactions extending the primer are carried out in the presence of a phosphatase enzyme, resulting in the cleavage of an alpha-beta phosphate bond and at least one additional phosphate bond of the incorporated nucleoside polyphosphates upon incorporation of the nucleoside monophosphate portion of the nucleoside polyphosphate. The phosphate bond cleavages resulting from the incorporation reactions are monitored with the electronic sensing elements to identify the types of nucleoside polyphosphates incorporated in the incorporation reactions, thus identifying the sequence of the plurality of template nucleic acids. In further embodiments, the two or more types of nucleoside polyphosphates comprise four types of nucleoside polyphosphates corresponding to the nucleobases A, G, T, and C, and in still further embodiments the electronic sensing elements sense ionic changes from the cleavage of the phosphate bonds.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

What is claimed:

1. A method of identifying a sequence of a plurality of template nucleic acids, said method comprising:
(a) providing a plurality of immobilized clonal populations of primed nucleic acid templates, each clonal population proximate to an electronic sensing element;
(b) exposing the plurality of immobilized clonal populations to a first type of nucleoside polyphosphate under conditions supporting a template directed incorporation of a nucleoside monophosphate portion of the first type of nucleoside polyphosphate; wherein the first type of nucleoside polyphosphate comprises a polyphosphate chain of three or more phosphates and a terminal blocking group, and wherein the incorporation reaction is carried out in the presence of a phosphatase enzyme and results in the cleavage of an alpha-beta phosphate bond and at least one additional phosphate bond of the incorporated nucleoside polyphosphate;
(c) electrically monitoring each of the clonal populations with the electronic sensing elements to detect whether one or more incorporations of the first type of nucleoside polyphosphate occurs at that clonal population;
(d) repeating steps (b) and (c) with second, third and fourth types of nucleoside polyphosphates,
wherein said repeating step (d) is conducted a number of times to thereby identify the sequence of the plurality of template nucleic acids.

2. The method of claim 1 wherein the electronic sensing elements sense ionic changes from the cleavage of the phosphate bonds.

3. The method of claim 1 wherein the electronic sensing elements sense pH changes from the cleavage of the phosphate bonds.

4. The method of claim 1 wherein the electronic sensing element comprises a field effect transistor (FET).

5. The method of claim 4 wherein the electronic sensing element comprises an ion sensitive field effect transistor (ISFET).

6. The method of claim 1 wherein the electronic sensing elements sense temperature changes resulting from the cleavage of the phosphate bonds.

7. The method of claim 1 wherein the clonal populations of primed nucleic acid templates are provided on beads.

8. The method of claim 1 wherein the clonal populations of primed nucleic acid templates are provided as separate regions on a substrate.

9. The method of claim 1 wherein the polyphosphate chain comprises between 3 and 20 phosphates.

10. The method of claim 1 wherein the polyphosphate chain comprises 3, 4, 5, 6, 7, 8, 9, 10, 11, or 12 phosphates.

11. The method of claim 1 wherein the first, second, third, and fourth types of nucleoside polyphosphates each correspond to a nucleobase independently selected from A, G, C, or T.

12. The method of claim 1, wherein the phosphatase enzyme comprises shrimp alkaline phosphatase.

13. The method of claim 1 wherein the terminal blocking group comprises a member selected from a methyl group, an amino hexyl group, a dye, an adduct, and a linker.

14. The method of claim 1 wherein the number of immobilized clonal populations of primed nucleic acid templates is between 1,000 and 10 million.

15. The method of claim 1 wherein the number of immobilized clonal populations of primed nucleic acid templates is between 100,000 and 5 million.

16. The method of claim 1 wherein cleavage of the at least one additional phosphate bond comprises cleavage of 2, 3, 4, 5, 6, 7, 8, 9, or 10 additional phosphate bonds.

17. The method of claim 1, wherein the second, third and fourth types of nucleoside polyphosphates comprise a polyphosphate chain of four or more polyphosphates.

18. The method of claim 1, wherein the electronic sensing elements sense changes in magnetic field caused by the cleavage of the phosphate bonds.

19. The method of claim 18, wherein the changes in magnetic field result from magnetic particles sensitive to changes in pH.

20. A method of identifying a sequence of a plurality of template nucleic acids, said method comprising:
(a) providing a plurality of single-molecule polymerase-template complexes, each complex comprising a template nucleic acid, a polymerase enzyme and a primer; wherein each complex is associated with an electronic sensing element;
(b) exposing the complexes to two or more types of nucleoside polyphosphates, wherein the two or more types of nucleoside polyphosphates each comprises a phosphate chain of three or more phosphates and a terminal blocking group, and wherein each type of nucleoside polyphosphate has a different number of phosphates; the exposing carried out under conditions supporting template dependent primer extension through multiple incorporation reactions, whereby the incorporation reactions extending the primer are carried out in the presence of a phosphatase enzyme resulting in the cleavage of an alpha-beta phosphate bond and at least one additional phosphate bond of the incorporated nucleoside polyphosphates; and
(c) detecting the phosphate bond cleavages resulting from the incorporation reactions with the electronic sensing elements to identify the types of nucleoside polyphosphates incorporated in the incorporation reactions to thereby sequence the plurality of template nucleic acids.

21. The method of claim 20 wherein the two or more types of nucleoside polyphosphates comprise four types of nucleoside polyphosphates corresponding to the nucleobases A, G, T, and C.

22. The method of claim 20 wherein the electronic sensing elements sense ionic changes from the cleavage of the phosphate bonds.

23. The method of claim 20 wherein the electronic sensing elements sense pH changes from the cleavage of the phosphate bonds.

24. The method of claim 20 wherein the electronic sensing element comprises a field effect transistor (FET).

25. The method of claim 24 wherein the electronic sensing element comprises an ion sensitive field effect transistor (ISFET).

26. The method of claim 20 wherein the electronic sensing elements sense temperature changes from the cleavage of the phosphate bonds.

27. The method of claim 20 wherein the polymerase enzyme is immobilized on a substrate.

28. The method of claim 27 wherein polymerase enzyme is immobilized in a zero mode waveguide.

29. The method of claim 20 wherein the polyphosphates of the nucleoside polyphosphates comprise between 3 and 20 phosphates.

30. The method of claim 20 wherein the polyphosphates of the nucleoside polyphosphates comprise 3, 4, 5, 6, 7, 8, 9, 10, 11, or 12 phosphates.

31. The method of claim 20 wherein the phosphatase enzyme comprises shrimp alkaline phosphatase.

32. The method of claim 20 wherein the terminal blocking group comprises a member selected from a methyl group, an amino hexyl group, a dye, an adduct, and a linker.

33. The method of claim 20 wherein the number of immobilized complexes is from 1,000 and 10 million.

34. The method of claim 20 wherein the number of immobilized complexes is from 100,000 and 5 million.

35. The method of claim 20 wherein cleavage of the at least one additional phosphate bond comprises cleavage of 2, 3, 4, 5, 6, 7, 8, 9, or 10 additional phosphate bonds.

36. The method of claim 20, wherein the detecting step (c) comprises detecting signals generated by the phosphate bond cleavages, wherein one or more characteristics of the signals are used to identify the type of nucleoside polyphosphates incorporated in the incorporation reactions.

37. The method of claim 20, wherein the electronic sensing elements sense changes in magnetic field caused by the cleavage of the phosphate bonds.

38. The method of claim 37, wherein the changes in magnetic field result from magnetic particles sensitive to changes in pH.

39. A method of identifying a sequence of a plurality of template nucleic acids, said method comprising:
(a) providing a plurality of immobilized single-molecule primed nucleic acid templates, wherein each single molecule template is proximate to an electronic sensing element;
(b) exposing the plurality of immobilized single molecules to a first type of nucleoside polyphosphate under conditions supporting a template directed incorporation of a nucleoside monophosphate portion of the first type of nucleoside polyphosphate and in the presence of a phosphatase enzyme; wherein the first type of nucleoside polyphosphate comprises a polyphosphate chain of three or more phosphates and a terminal blocking group; and whereby, upon incorporation, cleavage of the alpha-beta phosphate bond and cleavage of at least one additional phosphate bond of the polyphosphate chain occurs;
(c) electrically monitoring each of the single molecule templates with the electronic sensing elements to detect whether one or more incorporations of the type of nucleoside polyphosphate occurs at that single-molecule template;
(d) repeating steps (b) and (c) with second, third and fourth types of nucleoside phosphates,
wherein said repeating step (d) is conducted a number of times to thereby identify the sequence of the plurality of template nucleic acids.

40. The method of claim 39 wherein the electronic sensing elements sense ionic changes from the cleavage of the phosphate bonds.

41. The method of claim 39 wherein the electronic sensing elements sense pH changes from the cleavage of the phosphate bonds.

42. The method of claim 39 wherein the electronic sensing element comprises a field effect transistor (FET).

43. The method of claim 42 wherein the electronic sensing element comprises an ion sensitive field effect transistor (ISFET).

44. The method of claim 39 wherein the electronic sensing elements sense temperature changes resulting from the cleavage of the phosphate bonds.

45. The method of claim 39 wherein the single molecule primed nucleic acid templates are provided on beads.

46. The method of claim 39 wherein the single-molecule primed nucleic acid templates are provided as separate regions on a substrate.

47. The method of claim 39 wherein the polyphosphate chain comprises between 4 and 20 phosphates.

48. The method of claim 39 wherein the polyphosphate chain comprises 4, 5, 6, 7, 8, 9, 10, 11, or 12 phosphates.

49. The method of claim 39 wherein the first, second, third, and fourth types of nucleoside polyphosphates each correspond to a nucleobase independently selected from A, G, C, or T.

50. The method of claim 39 wherein the phosphatase enzyme comprises shrimp alkaline phosphatase.

51. The method of claim 39 wherein the terminal blocking group comprises a member selected from a methyl group, an amino hexyl group, a dye, an adduct, and a linker.

52. The method of claim 39 wherein the number of immobilized clonal populations of primed nucleic acid templates is between 1,000 and 10 million.

53. The method of claim 39 wherein the number of immobilized clonal populations of primed nucleic acid templates is between 100,000 and 5 million.

54. The method of claim 39 wherein cleavage of the at least one additional phosphate bond comprises cleavage of 2, 3, 4, 5, 6, 7, 8, 9, or 10 additional phosphate bonds.

55. The method of claim 39, wherein the second, third and fourth types of nucleoside polyphosphates comprise a polyphosphate chain of four or more polyphosphates.

56. The method of claim 39, wherein the electronic sensing elements sense changes in magnetic field caused by the cleavage of the phosphate bonds.

57. The method of claim 56, wherein the changes in magnetic field result from magnetic particles sensitive to changes in pH.

58. A method for increasing a signal from a template directed incorporation of a nucleoside monophosphate portion of a nucleoside polyphosphate, the method comprising:
(a) providing a plurality of immobilized clonal populations of primed nucleic acid templates, each clonal population proximate to an electronic sensing element;
(b) exposing the plurality of immobilized clonal populations to a first type of nucleoside polyphosphate under conditions supporting a template directed incorporation of a nucleoside monophosphate portion of the first type of nucleoside polyphosphate; wherein the first type of nucleoside polyphosphate comprises a polyphosphate chain of three or more phosphates and a terminal blocking group; and whereby, upon incorporation, cleavage of the alpha-beta phosphate bond and cleavage of at least one additional phosphate bond of the polyphosphate chain occurs, thereby generating a signal detectable by the electronic sensing elements;
(c) electrically monitoring each of the clonal populations with the electronic sensing elements to detect whether one or more incorporations of the type of nucleoside polyphosphate occurs at that clonal population by detecting the signal generated by cleavage of the alpha-beta phosphate bond and the at least one additional phosphate bond;
(d) repeating steps (b) and (c) with second, third and fourth types of nucleoside phosphates,
wherein the repeating step (d) is conducted a number of times to thereby identify the sequence of the plurality of template nucleic acids.

59. A method for increasing a signal from a template directed incorporation of a nucleoside monophosphate portion of a nucleoside polyphosphate, the method comprising:
(a) providing a plurality of single-molecule polymerase-template complexes, each complex comprising a template nucleic acid, a polymerase enzyme and a primer; wherein each complex is associated with an electronic sensing element;
(b) exposing the complexes to two or more types of nucleoside polyphosphates, wherein the two or more types of nucleoside polyphosphates each comprises a phosphate chain of three or more phosphates, and wherein each type of nucleoside polyphosphate has a different number of phosphates and a terminal blocking group; the exposing carried out under conditions supporting template dependent primer extension through multiple incorporation reactions, whereby the incorporation reactions extending the primer are carried out in the presence of a phosphatase enzyme resulting in the cleavage of an alpha-beta phosphate bond and at least one additional phosphate bond of the incorporated nucleoside polyphosphates, thereby generating a signal detectable by the electronic sensing elements; and
(c) detecting the signals from the phosphate bond cleavages resulting from the incorporation reactions with the electronic sensing elements to identify the types of nucleoside polyphosphates incorporated in the incorporation reactions to thereby sequence the plurality of template nucleic acids.

60. A method for identifying a sequence of a plurality of template nucleic acids, said method comprising:
(a) providing a plurality of immobilized clonal populations of nucleic acids, wherein each clonal population is proximate to an electronic sensing element;
(b) exposing the plurality of immobilized clonal populations to a first type of nucleoside polyphosphate under conditions supporting a template directed incorporation of a nucleoside monophosphate portion of the first type of nucleoside polyphosphates into primers hybridized to the nucleic acids; wherein the first type of nucleoside polyphosphate comprises a polyphosphate chain of three or more phosphates and a terminal blocking group; and whereby, upon incorporation, cleavage of the alpha-beta phosphate bond and cleavage of at least one additional phosphate bond of the polyphosphate chain occurs, thereby releasing at least three hydrogen ions;
(c) electrically monitoring each of the clonal populations with the electronic sensing elements to detect whether one or more incorporations of the first type of nucleoside polyphosphate occurs at that clonal population by detecting the released hydrogen ions at that clonal population;
(d) repeating steps (b) and (c) with second, third and fourth types of nucleoside phosphates,
wherein the repeating step (d) is conducted a number of times to thereby identify the sequence of the plurality of template nucleic acids.

61. A method for identifying a sequence of a plurality of template nucleic acids, the method comprising:
(a) providing a plurality of immobilized clonal populations of primed nucleic acid templates, each clonal population proximate to an electronic sensing element;
(b) exposing the plurality of immobilized clonal populations to a first type of nucleoside polyphosphate under conditions supporting a template directed incorporation of a nucleoside monophosphate portion of the first type of nucleoside polyphosphate; wherein the first type of nucleoside polyphosphate comprises a polyphosphate chain of three or more phosphates and a terminal blocking group; and whereby, upon incorporation, cleavage of the alpha-beta phosphate bond and cleavage of at least one additional phosphate bond of the polyphosphate chain occurs, thereby generating a byproduct detectable by the electronic sensing element;
(c) electrically monitoring each of the clonal populations with the electronic sensing elements to detect whether one or more incorporations of the type of nucleoside polyphosphate occurs at that clonal population by detecting the byproduct generated by the cleavage of the phosphate bonds;
(d) repeating steps (b) and (c) with second, third and fourth types of nucleoside phosphates,
wherein the repeating step (d) is conducted a number of times to thereby identify the sequence of the plurality of template nucleic acids.
